(19) United States
(12) Patent Application Publication
(10) Pub. No.: US 2026/0345661 A1
(43) Pub. Date: Nov. 5, 2026

(54) IMPROVED GLYCEMIC CONTROL BY ADMINISTRATION OF MICRO-RNA 192

(71) Applicant: THE REGENTS OF THE UNIVERSITY OF CALIFORNIA, Oakland, CA (US)

(72) Inventors: Marisa Medina, Oakland, CA (US); Ronald Krauss, Oakland, CA (US)

(21) Appl. No.: 19/104,096

(22) PCT Filed: Aug. 16, 2023
(86) PCT No.: PCT/US2023/072327

§ 371 Date: Feb. 14, 2025

Related U.S. Application Data
(60) Provisional application No. 63/371,618, filed on Aug. 16, 2022.

(51) Int. Cl. A61K 31/7105; A61K 45/06; A61P 3/10

(52) U.S. Cl. CPC A61K 31/7105; A61K 45/06; A61P 3/10

(57) ABSTRACT

The disclosure provides, in various aspects, methods of achieving a selected therapeutic outcome in a subject by administration to the subject of a therapeutically effective amount of an miR-192 agent. In further aspects, the disclosure provides methods of assessing an enhanced risk of statin-induced new onset diabetes (NOD) in a subject, as well as kits comprising elements to perform methods of the disclosure.

### CROSS-REFERENCE TO RELATED APPLICATIONS

This application claims the priority benefit under 35 U.S.C. § 119(e) of U.S. Provisional Application No. 63/371,618, filed Aug. 16, 2022, which is incorporated herein by reference in its entirety.

### STATEMENT REGARDING FEDERALLY SPONSORED RESEARCH OR DEVELOPMENT

This invention was made with government support under grant no. P50 GM115318 awarded by The National Institutes of Health. The government has certain rights in the invention.

### INCORPORATION BY REFERENCE OF MATERIALS SUBMITTED ELECTRONICALLY

This application contains, as a separate part of the disclosure, a Sequence Listing in computer readable form (Filename: 50030_SeqListing.xml; Size: 10,122 bytes; Created: Aug. 16, 2023), which is incorporated by reference in its entirety.

### BACKGROUND

In the body, normal function relies on the tight regulation of circulating glucose levels. This glycemic control is primarily regulated by two incretin hormones: glucose-dependent insulinotropic polypeptide (also known as gastric inhibitory polypeptide), referred to as GIP; and Glucagon-like peptide-1, referred to as GLP1. Food intake stimulates the release of these hormones from the intestine. Upon binding their respective receptors in pancreatic β-cells, insulin is released and reduces blood glucose levels by promoting various glucose uptake processes. The mechanisms of this glucose-stimulated insulin secretion (GSIS) are essential to maintenance of cellular function and overall health.

### SUMMARY OF THE INVENTION

In subjects having Type II diabetes (T2DM), the GIP receptor, GIPR, becomes insensitive, resulting in loss of GSIS. Meanwhile, GLP1 signaling remains largely intact and provides a therapeutic target for promoting GSIS in diabetic subjects. Two classes of agents directed to this pathway are currently administered to treat T2DM. First, GLP1R agonists, for example, drugs such as exenatide and liraglutide, are GLP1 molecular mimics that strongly activate GLP1R. A second class of agents targeted to the GLP1 pathway are dipeptidyl-peptidase-IV (DPP-IV) inhibitors, for example, drugs such as sitagliptin and vildagliptin. Circulating GLP1 is subject to rapid enzymatic decay by DPP-IV, and inhibitors thereof stabilize GLP1 against rapid clearance from the system, thus increasing GLP1R activity.

Both of the foregoing classes of agents are generally effective in promoting GSIS and preventing pathologies associated with T2DM, such as cardiovascular disease. However, despite the physiological benefits imparted by these drugs, clinical adoption has been limited by their poor tolerability for many subjects. Detrimental side effects are common, for example, severe gastrointestinal issues such as nausea, diarrhea, and vomiting.

Accordingly, there is a need in the art for treatments that potentiate, or otherwise augment the activity of GLP1R agonists or DPP-IV inhibitors.

Meanwhile, another condition wherein GSIS is impaired is statin-induced new onset diabetes (NOD). Statins, also known as HMG-CoA reductase inhibitors, are a class of lipid-lowering or hypolipidemic drugs that have achieved widespread adoption for their ability to prevent cardiovascular disease in at-risk subjects. However, it has been observed by various research groups that statin use increases the probability of developing new-onset diabetes (NOD), prompting the Food and Drug Administration to add information to statin labels regarding this increased risk. The physiological mechanisms by which statins induce diabetes are not known. While it is believed that the risk of new-onset of diabetes does not outweigh the benefits of statins on cardiovascular disease risk, the widespread and long-term use of these drugs by millions of users means many potential cases of NOD. Accordingly, there is a need in the art for understanding the cause of NOD in statin users and developing preventative measures.

As detailed in the Examples section below, the inventors of the present disclosure have made numerous beneficial discoveries regarding GLPR1-mediated control of GSIS. Particularly, the inventors of the present disclosure have established that micro-RNA 192 (miR-192) is a novel inducer of GLP1R that enhances GSIS and improves glycemic control. Furthermore, the inventors of the present disclosure have demonstrated interventions in animal subjects with impaired GSIS wherein miR-192 promoted increased GLP1 activity and insulin release. Accordingly, the present disclosure provides the art with various new and useful inventions as disclosed herein.

Specifically, as detailed herein, the inventors of the present disclosure have, by substantial experimentation, determined that in certain subjects, statin use induces a deficit in miR-192 abundance. MiR-192, is a conserved micro-RNA expressed in the intestine, colon and other sites and which is found in circulation. The inventors of the present disclosure have determined that impairment of this regulatory molecule in a subset of individuals was associated with statin-induced dysglycemia. By various experiments, the inventors of the present disclosure have determined that administered miR-192 can rescue statin-induced impairment of glucose-stimulated insulin secretion. Additional work demonstrates that administered miR-192 enhances GLP-1R expression and promotes GLP1 potentiated GSIS beyond the context of statin, and that these therapeutic effects may be recapitulated in diabetes subjects in general.

The foregoing discoveries provide the art with numerous novel and useful inventions, as described in detail below.

In a first aspect, the scope of the invention provides a method of increasing GLP1-mediated GSIS in subjects having dysregulated GSIS. In one embodiment, the scope of the invention encompasses methods of increasing GLP1R expression and abundance in β-cells. In one embodiment, the scope of the invention encompasses methods of increasing GLP1 activity in β-cells. In one embodiment, the scope of the invention encompasses methods of increasing the activity of GLP1 agonists and DPP-IV inhibitors. In one embodiment, the scope of the invention encompasses prevention and treatment of diabetes, for example, T2D diabetes. In one embodiment, the scope of the invention encompasses methods of treating and preventing NOD associated with statin use.

In a second aspect, the scope of the invention encompasses novel diagnostic methods for assessing susceptibility to diabetes, including statin-induced diabetes.

In some aspects, the disclosure provides a method of achieving a selected therapeutic outcome in a subject by administration to the subject of a therapeutically effective amount of an miR-192 agent. In some embodiments, the miR-192 agent comprises an miR-192 nucleic acid sequence, sequence variant thereof, or chemical mimic thereof. In further embodiments, the miR-192 agent comprises at least 90% sequence identity to hsa-miR-192-5p (SEQ ID NO: 1). In some embodiments, the nucleic acid is packaged in an extracellular vesicle. In some embodiments, the miR-192 agent comprises a transformation vector comprising an miR-192 nucleic acid sequence. In further embodiments, the transformation vector expresses a sequence comprising at least 90% sequence identity to hsa-miR-192-5p (SEQ ID NO: 1). In various embodiments, the miR-192 agent is administered systemically. In some embodiments, the selected therapeutic outcome is increasing GSIS. In some embodiments, the selected therapeutic outcome is increasing GLP1 activity. In further embodiments, the selected therapeutic outcome is increasing GLP1R abundance and/or activity. In still further embodiments, the selected therapeutic outcome is weight loss. In some embodiments, the selected therapeutic outcome is treatment of Type II diabetes. In further embodiments, the selected therapeutic outcome is the prevention of new onset diabetes. In still further embodiments, the subject is a statin user. In some embodiments, the selected therapeutic outcome is treatment or prevention of a cardiovascular disease. In further embodiments, the cardiovascular disease is atherosclerotic cardiovascular disease. In some embodiments, the MiR-192 agent is co-administered with a GLP-1 agonist or DPP-IV inhibitor.

In some aspects, the disclosure provides a method of assessing elevated risk of statin-induced NOD in a subject, comprising the steps: a first sample is obtained from a subject that is not using statins; miR-192 family member abundance in the sample is measured; a second sample is obtained from the subject after they have been administered statins; miR-192 family member abundance in the sample measured; the first and second measurements of miR-192 family member abundance are compared; and risk of statin-induced NOD is assessed, wherein a decrease in miR-192 family member abundance after statin use is indicative of elevated risk of statin-induced NOD.

In further aspects, the disclosure provides a method of assessing elevated risk of developing statin-induced NOD in a subject, comprising the steps: a cellular sample is obtained from a subject; a cell culture derived from the sample is established; miR-192 family member abundance in the cells is measured in the absence of statins; one or more selected statins are applied to the cell culture; miR-192 family member abundance in the cells is measured after the application of statins; the first and second measurements of miR-192 family member abundance are compared; and risk of statin-induced NOD is assessed, wherein a decrease in miR-192 family member abundance in the cells in response to statin use is indicative of elevated risk of statin-induced NOD.

In some aspects, the disclosure provides a kit comprising elements for the performance of a method of the disclosure.

The various inventions of the present disclosure are described in detail next.

### BRIEF DESCRIPTION OF THE DRAWINGS

FIG. 1 shows plasma lipid measurements of statin-induced NOD cases and controls. Pre-statin values were calculated as the average values in the 3 years prior to statin initiation. Percent changes were calculated as difference of the average on-statin vs. pre-statin measures. *p<0.05

FIG. 2 shows the identification of MIR192 and MIR194 as putative factors underlying new-onset Type 2 diabetes. (A) Change in transcript levels in iPSCs derived from statin users who developed new-onset diabetes (NOD cases, n=24), or maintained normal glycemic control (controls, n=24) after 24 hr incubation with 500 nM simvastatin, 250 nM atorvastatin or mock buffer. Values were variance stabilized (~log 2 transformation) in DESeq2 and deltas were calculated as statin-control expression levels. (B) Schematic of the MIR194-2HG, which encompasses two miRNAs, MIR192 and MIR194.

FIG. 3 shows that MIR192-5p mimic upregulates GLP1R in INS-1 cells and MIR192-5p inhibitor reversed the effect. INS-1 cells were transfected with MIR192-5p mimic, MIR192-5p mimic plus MIR192-5p inhibitor, or their respective controls. MIR192-5p (A), Glp1r transcript (B), GLP1R protein (C) were quantified by qPCR and western blot analysis. Similarly, INS-1 cells were transfected with MIR204-5p mimic, MIR204-5p inhibitor, or their respective controls, and Glp1r transcript (D) and GLP1R protein (E) were quantified by qPCR and western blot analysis. (F) A schematic presentation of the sequence predicted to be targeted by MIR192-5p in wild type and mutant human GLP1R 3′UTR luciferase constructs. (G) INS-1 cells were co-transfected with luciferase constructs containing the GLP1R 3′ UTR both intact and after mutagenesis of the predicted MIR192-5p binding site along with the MIR192-5p mimic, MIR192-5p mimic plus inhibitor, or corresponding scrambled controls. Luciferase levels were compared to empty vector (EV) and GAPDH 3′ UTR negative controls and an SCD 3′ UTR positive control. (H) Positive correlation observed between transcript levels of MIR194-2HG and GLP1R in 34 iPSCs quantified by RNAseq with values variance stabilized and quantile normalized. *p<0.05, **p<0.01, ***p<0.01.

FIG. 4 shows MIR192-5p mimic increases glucose-stimulated insulin secretion (GSIS). INS-1 cells were transfected with MIR192-5p mimic, MIR192-5p mimic plus MIR192-5p inhibitor, or their respective controls for 48 hours, and GSIS was conducted with A) 17.8 mM glucose with 50 nM GLP-1 for 30 min, B) 16 mM glucose with or without 100 nM exendin-4 for 30 min, or C) 17.8 mM glucose with 100 nM exendin-4, plus 0, 500, or 1000 nM simvastatin for 30 min. Media insulin levels were measured with ELISA and expressed as fold change compared to control.

FIG. 5 depicts a hypothetical model.

FIG. 6 shows the change of miRNA levels in iPSCs after 24 hr statin exposure.

FIG. 7 shows A) MIR192-5p upregulates Glp1r in bTC3 cells. B) MIR-194-5p and MIR-215-5p mimics upregulate GLP1R protein levels in INS-1 cells compared to control mimic. C) MIR194-5p and MIR215-5p mimics upregulate luciferase reporters containing the GLP1R 3′ UTR or the mutated GLP1R 3′UTR.

FIG. 8 shows results of experiments in which INS-1 cells were transfected with the miR192 mimic or a scrambled control, stimulated for 30 minutes with glucose (3 mM or 17.8 mM) and a GLP1R agonist (50 nM GLP-1 or 25 mM exendin-4) or GLP-1R antagonist (100 mM exendin9-39), and insulin in the media was quantified by ELISA. N=6-12/treatment. B-D) Primary murine islets from wildtype and GLP1R/GIPR double knockout animals were transfected with miR192 mimic or control, reaggregated and (B) miR192 and Glp1r transcript levels were quantified by qPCR and normalized to miR16 and Actb, C) GLP-1R protein was visualized in WT islets through incubation with LUXendin-645 (a GLP-1R fluorescent probe) and DAPI, and D) GSIS was quantified by perifusion. *p<0.05, **p<0.01, ***p<0.001, ****p<0.0001.

FIG. 9 depicts a model of miR192 mediated effects of statin in individuals who develop NOD vs. those who maintain normoglycemia.

FIG. 10 shows results of experiment in which A) INS-1 cells were pre-incubated with 3.0 mM glucose (non-stimulatory) and 500 μM simvastatin, 250 μM atorvastatin or mock for 30 minutes, after which cells were treated with 17.8 mM glucose and 50 nM GLP-1. Media insulin was quantified by ELISA and normalized to cellular protein (n=6). B) INS-1 cells were transfected with the miR192 mimic, mimic+inhibitor, or a ser control for 24 hours, after which cells were treated with simvastatin or a mock buffer in either 17.8 mM glucose+50 nM GLP-1 or 3 mM glucose+50 nM GLP-1. After 30 minutes media insulin was quantified. Values were calculated as a fold change from the mock treatment. N=3-6/condition. *p<0.05, **p<0.01 from mock with 17.8 mM glucose.

FIG. 11 shows A) ChIP-seq signal of POLR2A at the MIR194-2HG promoter in various tissues from ENCODE. 1000 is the maximal signal. B) Caco2 and HepG2 cells were treated with varying concentrations of glucose and miR192 in the culture media was by qPCR after 24 hr. C) Plasma miR192 was quantified in blood concentration from the jugular vein of male C57BL/6J mice at baseline and from the portal vein 15 minutes after an oral bolus of glucose (2 g/kg), n=7. D) Women were fed a standardized meal, and blood was collected with a DPP-IV inhibitor for 5 hr after the meal. GLP-1 was quantified by electrochemiluminescence. miR192 was quantified by qPCR, normalized to levels of miR16 and expressed as a fold change from time 0. N=16. E) Model of how intestine and/or liver derived miR192 can impact β-cell function. F) HepG2 were treated with 25 mM glucose or control for 24 hr after which miR192 was quantified in isolated exosomes, the exosome-free media and within the cells. G) INS-1 cells were incubated with exosomes from 25 mM glucose treated HepG2 or Caco2 cells, or media with exosome-free FBS, and after 24 hr cellular miR192 were quantified. All culturing was performed in media supplemented with exosome-free FBS. *p<0.05, **p<0.01

FIG. 12 shows results of experiments in which C57BL6/J male and female mice were fed a simvastatin supplemented or chow diet to create a model of statin induced NOD. Plasma non-HDLC and miR192 in intestine and plasma after 4 weeks of diet, n=3-12/sex/treatment. Sexes combined for miR192 levels. *p<0.05, **p<0.01

FIG. 13 shows results of experiments in which INS-1 cells were incubated with 0, 50 or 100 μg protein equivalent ExoGlow-labeled exosomes isolated from HepG2 conditioned media. i) Representative images were taken after 5 days of incubation showing dose dependent internalization of labeled exosomes by INS-1. ii) Summary of fluorescent quantitation in all cells imaged.

FIG. 14 shows results of experiments in which C57BL6/J male mice fed a GAN diet were injected with either miR192 AAV (n=4) or control AAV (n=5). After 4 weeks, i) GTT was performed with 1.5 g/kg glucose after a 6 hour fast, and plasma glucose was determined at 0, 15, 30, 60, 90, and 120 minutes. ii) Pancreatic miR192 was quantified by qPCR. Mean±SEM, *p<0.05, ***p<0.001

### DETAILED DESCRIPTION OF THE INVENTION

The scope of the invention encompasses a general method as follows:

A method of promoting GSIS in β-cells of a subject, by administration to the subject of a therapeutically effective amount of an miR-192 agent.

The scope of the invention encompasses various embodiments of the foregoing general method as described herein.

### Part I. MIR-192 Agents

### miR-192 Agent

The scope of the invention encompasses the administration of an miR-192 agent to a subject. As used herein, an “miR-192 agent” comprises any composition of matter that: is or comprises an miR-192 family member nucleic acid sequence or variant or mimic thereof; a composition that is processed in the body to become an miR-192 family member nucleic acid sequence or variant or mimic thereof; or composition that induces the expression of an miR-192 family member nucleic acid sequences or variants thereof in the body.

References to miR-192 family members encompass miR-192, miR-194, and miR-215. References to miR-192 family members made herein will encompass human sequences, artificial sequences based thereon, and homologs and orthologs of the human sequence as found in other mammalian species. For ease of reference, sequences disclosed herein will be human sequences, but it will be understood that sequences from other species are encompassed as well, for example, to the extent such sequences elicit the desired biological effect in the subject to which it is administered.

In a first aspect, the miR-192 agent may comprise an active miR-192, miR-194, or miR-215 sequence, a variant thereof, or a mimic thereof. In this implementation, the miR-192 agent is a composition of matter that is miR-192, miR-194, miR-215, or is a nucleic acid sequence or other composition of matter that is of sufficient chemical similarity to the miR-192, miR-194, or miR-215 molecules to recapitulate the biological activity of miR-192 in target cells, e.g., β-cells.

In a first embodiment, the miR-192 agent is miR-192-5p. In one embodiment, the miR-192 is a sequence comprising at least 90%, at least 95%, at least 99%, or 100% sequence identity to hsa-MiR-192-5p: CUGACCUAUGAAUUGACAGCC (SEQ ID NO: 1). In one embodiment, the miR-192 is a sequence comprising at least 90%, at least 95%, at least 99%, or 100% sequence identity to hsa-MiR-192-3p: CUGCCAAUUCCAUAGGUCACAG (SEQ ID NO: 2). In one embodiment, the miR-192 agent comprises a miR-192 pri-mRNA, for example, a sequence comprising at least 90%, at least 95%, at least 99%, or 100% sequence identity to hsa-pri-miR-192: sequence. In one embodiment, the miR-192 agent comprises a miR-192 pre-mRNA, for example, a sequence comprising at least 90%, at least 95%, at least 99%, or 100% sequence identity to the hsa-pre-miR-192 sequence:

(SEQ ID NO: 8)
GCCGAGACCGAGUGCACAGGGCUCUGACCUAUGAAUUGACAGCCAGUGCU
CUCGUCUCCCCUCUGGCUGCCAAUUCCAUAGGUCACAGGUAUGUUCGCCU
CAAUGCCAGC.

In another embodiment, the miR-192 agent is miR-194. In one embodiment, the miR-194 is a sequence comprising at least 90%, at least 95%, at least 99%, or 100% sequence identity to hsa-miR-194-5p: uguaacagcaacuccaugugga (SEQ ID NO: 3). In one embodiment, the miR-192 is a sequence comprising at least 90%, at least 95%, at least 99%, or 100% sequence identity to hsa-miR-194-3p: ccaguggggcugcuguuaucug (SEQ ID NO: 4). In one embodiment, the miR-192 agent comprises a miR-194 pri-mRNA, for example, a sequence comprising at least 90%, at least 95%, at least 99%, or 100% sequence identity to hsa-pri-miR-194. In one embodiment, the miR-192 agent comprises a miR-194 premRNA, for example, a sequence comprising at least 90%, at least 95%, at least 99%, or 100% sequence identity to the hsa-pre-miR-194. In some embodiments, the miR-192 agent comprises a miR-194 premRNA, for example, a sequence comprising at least 90%, at least 95%, at least 99%, or 100% sequence identity to the hsa-pre-miR-194-2 sequence:

(SEQ ID NO: 9)
UGGUUCCCGCCCCCUGUAACAGCAACUCCAUGUGGAAGUGCCCACUGGUU
CCAGUGGGGCUGCUGUUAUCUGGGGCGAGGGCCAG.

In another embodiment, the miR-192 agent is miR-215. In one embodiment, the miR-192 is a sequence comprising at least 90%, at least 95%, at least 99%, or 100% sequence identity to hsa-miR-215-3p: ucugucauuucuuuaggccaaua (SEQ ID NO: 5). In one embodiment, the miR-192 agent comprises a miR-215 pri-mRNA, for example, a sequence comprising at least 90%, at least 95%, at least 99%, or 100% sequence identity to hsa-pri-miR-215. In one embodiment, the miR-192 agent comprises a miR-215 pre-mRNA, for example, a sequence comprising at least 90%, at least 95%, at least 99%, or 100% sequence identity to the hsa-pre-miR-215 sequence:

(SEQ ID NO: 10)
AUCAUUCAGAAAUGGUAUACAGGAAAAUGACCUAUGAAUUGACAGACAAU
AUAGCUGAGUUUGUCUGUCAUUUCUUUAGGCCAAUAUUCUGUAUGACUGU
GCUACUUCAA.

In one embodiment, the miR-192 agent comprises a variant of an miR-192, miR-194, or miR-215 sequence, for example, a nucleic acid sequence comprising one, two, three, four, or more modifications of the native sequence, including, for example, base substitutions, insertions, deletions, etc., for example, comprising sequence modifications that substantially retain the biological activity of miR-192, miR-194, or miR-215 parent sequences on which they are based, for example, in some cases enhancing the activity of the parent sequence. In various embodiments, the miR-192 variant comprises at least 90%, at least 95%, or at least 99% sequence identity to a parent miR-192, miR-194, or miR-215 sequence, for example, miR-192-5p, for example, hsa-MiR-192-5p.

The miR-192 agents of the invention further encompass miR-192 mimics, which comprise chemical analogs of miR-192, miR-194, or miR-215 sequences, for example, a hsa-miR-192-5p sequence. The chemical analogs may comprise modifications or substitutions of the nucleotide sugar backbone, modifications of the nucleobase element of the nucleotide, or modifications of the linkage between nucleotides. These modifications may extend the biostability of or half-life of the composition, facilitate delivery, or improve the biological efficacy of the molecule. In various embodiments, the miR-192 agents of the invention may comprise DNA, RNA, or nucleoside analogs and modified forms thereof. In some embodiments, the miR-192 agent comprises a peptide nucleic acid, for example N-(2aminoethyl)-glycine. In some implementations, the miR-192 mimic comprises one or more nucleotides having 2′-O-methyl (2′-O-Me), 2′-O-methoxyethyl(2′-MOE) or 2′-fluoro (2′-F) modified sugar moieties of one or more nucleotides, which confers increased nuclease resistance and may improve affinity for target gene sequences. In some embodiments, the miR-192 mimic comprises a locked nucleic acid (LNA), or bridged nucleic acid (BNA), wherein the ribose moiety of one or more bases is modified with an extra bond or “bridge” connecting the 2′ oxygen and 4′ carbon. In one embodiment, the miR-192 mimic comprises an miR-192 sequence comprising a terminal chemical modifications, for example, a Cy3-, cholesterol-, biotin- or amino-modified oligonucleotide.

In one embodiment, the miR-192 mimic is miRVana™ miRNA mimic by Invitrogen Biosciences, Inc., catalog number #4464066.

### Vesicular Delivery

Native cells, cancer cells, and pathogens utilize various vesicles to achieve release of micro-RNAs and their subsequent delivery to target cells. As set forth in the examples, the inventors of the present disclosure herein demonstrate effective therapeutic effects by the use of miR-192 agents packaged in vesicles. Accordingly, in one implementation, the scope of the invention encompasses an miR-192 agent packaged or contained in a vesicle.

In one implementation, the vesicle containing miR-192 agent is an exosome. Exosomes comprise membrane-bound extracellular vesicles secreted by various cell types, typically having a size range of 40-120 nm in diameter. Exosomes are used by cells for intercellular communication by transfer of bioactive molecules such as miRNAs. In a related implementation, the vesicle is a microvesicle, also known as a shedding vesicle, for example, in the size range of 100-1000 nm, comprising membrane bound vesicles that are shed from certain cell types. Delivery of miRNAs within exosomes or other vesicles protects the miRNAs within from degradation by nucleases or other destructive factors. Exogenously delivered exosomes generally have low toxicity and low immunogenicity.

In one implementation, the exosomes or other vesicles are produced exogenously, for example, from cultured cells. In one embodiment, the cultured cells are autologous cell cultures derived from the subject's own cells. In one embodiment, the cell cultures are allogenic cell cultures derived from compatible cell sources.

In an alternative embodiment, the exosome or other vesicle is produced endogenously, for example from transduced cells within the subject engineered to produce miR-192 molecules and secrete them into the general circulation or pancreatic compartment.

Transformation vectors. In one implementation, the miR-192 agent comprises a transformation vector which transforms target cells in the subject to express an miR-192 sequence. In various embodiment, the expression vector may express a sequence comprising at least 90%, at least 95%, at least 99%, or 100% sequence identity to a selected miR-192 sequence, for example, an miR-192 gene, an miR-192 pre-RNA sequence, an miR-192-pri-RNA sequence, or hsa-miR-192-5p.

The transformation vector may comprise a coding nucleic acid sequence under control of a suitable promoter, for example, a constitutive promoter, an inducible promoter, and/or a cell-specific promoter. For example, the insulin gene INS promoter has been used to direct islet β-cell specific expression of transgenes.

Exemplary transformation vectors comprise viral and non-viral systems. Exemplary viral platforms include adenoviruses, vaccinia viruses, adeno-associated viruses, lentivirus, retroviruses, and herpes virus. In one implementation, the transformation vector is an element of a CRISPR-Cas9 or like system for the targeted knock-in of genes. The transformation vector may be delivered by suitable means, including systemic delivery by intravenous injection or infusion, subcutaneous or intraperitoneal delivery, or by localized injection or implantation at the target site or in proximity thereto. The delivery may be by biolistic methods, electroporation, sonoporation, magnetofection, and chemical or lipid based transfection agents.

The transformation vector may be delivered and/or targeted to a particular cell compartment, or may be configured for selective expression only in a certain cell type. in one embodiment, the transformation vector is delivered to the pancreas or the β-cells and/or is configured for selective targeting to or expression in pancreatic cells or islet β-cells. For example, targeted expression of a therapeutic gene in the islet β-cells of the pancreas has been demonstrated, for example, as described in Erendor et al., 2021. Lentivirus Mediated Pancreatic Beta-Cell-Specific Insulin Gene Therapy for STZ-Induced Diabetes, Molecular Therapy 29: P149-161. Retrograde pancreatic intraductal delivery of AAV vectors has been demonstrated, for example, in Quirin et al., 2018. Safety and Efficacy of AAV Retrograde Pancreatic Ductal Gene Delivery in Normal and Pancreatic Cancer Mice, Molecular Therapy 8:8-20.

In another embodiment, the inventors of the present disclosure have advantageously determined that the miR-192 agents of the invention are able to act upon the islet beta cells when present in the general circulation. Accordingly, in one embodiment the scope of the invention encompasses transformation vectors that act in readily transformed non-pancreatic cells to produce miR-192 sequences that are subsequently carried to the pancreas by the circulatory system. In one embodiment, the transformation vector comprises signals or other elements that facilitate miR-192 packaging in vesicles, such as exosomes, and their release into the general circulatory system.

Formulations and Pharmaceutical Compositions. The methods and compositions of the invention encompass the administration of miR-192 agents by various methods. The miR-192 agents may be formulated in what will be termed “pharmaceutical compositions.” As used herein, a pharmaceutical composition will comprise one or more miR-192 agents and may further comprise any number of additional compositions of matter, including excipients, carriers, diluents, release formulations, drug delivery or drug targeting vehicles, as well as additional active therapeutic agents. The pharmaceutical compositions of the invention may be formulated to be compatible with the selected route of administration.

The pharmaceutical compositions of the invention may comprise one or more drug delivery compositions. Drug delivery compositions encompass any moieties, materials, or other compositions of matter that facilitate the delivery of the miR-192 agent to islet β-cells or other target cells. The pharmaceutical compositions may encompass any form of combination, including functionalization of the miR-192 agent with the delivery composition, conjugation of the miR-192 agent to the delivery composition; admixture of the miR-192 agent with the delivery composition; encapsulation or infusion of the miR-192 agent within the delivery composition, or any other combination.

In one implementation, the pharmaceutical compositions comprise carriers. Exemplary carriers include: liposomes; extracellular vesicles or synthetic mimetics thereof, such as exosomes; red blood cells modified with an miR-192 agent; microspheres, such as poly(lactic-co-glycolic acid) (PLGA) microspheres; and other drug delivery nanoparticles such as PLGA-PEG nanoparticles, alginate or chitosan nanoparticles, silica nanoparticles, and iron oxide nanoparticles.

Targeting Moieties. The pharmaceutical compositions of the invention may comprise targeting moieties that facilitate delivery of the miR-192 to the pancreas, for example, to the islet β-cells thereof. Targeting moieties may comprise peptides, small molecules, or other species that selectively deliver to the pancreas.

In one embodiment, the miR-192 agent is formulated as a drug-antibody conjugate, for example, comprising an antibody or antigen-binding fragment thereof targeted to a ligand present in the pancreas, for example, the islet β-cells thereof. For example, as demonstrated in Jeong et al., 2005, Anti-GAD antibody targeted non-viral gene delivery to islet beta cells, Journal of Controlled Release 107:562-570, nucleic acids in polymeric delivery vehicle were efficiently delivered to islet cells by conjugation to an antibody fragment targeting the glutamic acid decarboxylase (GAD), a major islet cell antigen.

In one embodiment, the miR-192 agent is conjugated to a ligand that selectively targets it to the pancreas. For example, previous work in the field has shown that ligand activation of GLP1R leads to rapid internalization of the ligand, wherein agents fused to GLP1 receptor binding moieties can be targeted to the islet cells, for example, as described in Ammala et al., 2018, Targeted delivery of antisense oligonucleotides to pancreatic β-cells, Science Advances 4: eaat3386 wherein it was demonstrated that therapeutic nucleic acids conjugated by disulfide bonds to GLP1 sequences could be delivered to islet cells.

Implants and Drug Eluting Structures. In one embodiment the delivery composition comprises or is incorporated within an implant, for example, a drug-eluting implant placed within the target tissue, for example, the pancreas or ducts in connection therewith. Exemplary implants include, for example, polymeric drug-eluting wafers, injectable hydrogels, implantable hydrogel scaffolds, and other drug-eluting implants known in the art. Transdermal patches, micro-pumps, and other drug delivery devices known in the art may be used.

Combination Products. The methods of the invention will be understood to further encompass the combined administration of an miR-192 agent and one or more additional active agents for treatment of diabetes or other condition. Combined administration, as used herein may encompass any combination of an miR-192 agent and a secondary agent, for example, a GLP-1R agonist or DPP-IV inhibitor, as described below. In one embodiment, the first and second treatments are administered in a combination product, comprising an miR-192 agent and an additional agent, for example, administered in a single dosage form or co-packaged.

Dosage Forms. The pharmaceutical compositions of the invention may be formulated in any number of dosage forms. Exemplary dosage forms include: liquid solutions; sachets; capsules; tablets; solids or granules; suspensions in a liquid; emulsions; aqueous and non-aqueous solutions; isotonic sterile injection solutions; compositions stored in a freeze-dried, lyophilized condition; and other dosage forms known in the art.

### Part II. Methods of Treatment

Methods of Prevention and Treatment. Various implementations of the general method of the invention are encompassed by the present disclosure. Certain embodiments are directed to treatment of a selected condition. As used herein, “treatment” will encompass any positive therapeutic outcome with respect to an enumerated condition. In the context of diabetes or impaired GSIS, treatment will include, for example, improving GSIS, reducing or inhibiting hyperglycemia, maintaining normal blood glucose concentrations (for example, maintaining blood glucose at less than 130 mg/dl when fasting), and any other reduction, amelioration, or inhibition of symptoms of the selected condition. In the case of cardiovascular disease, treatment will include, for example, prevention of: new or recurrent myocardial infarction, stroke, transient ischemic attack, peripheral vascular disease, unstable angina, coronary artery revascularization procedures, percutaneous coronary intervention, heart failure, and/or cardiovascular death.

Certain methods of the invention are directed to prevention of a selected condition. As used herein, “prevention” of an enumerated condition means any inhibition of the progression of the condition. For example, in the context of diabetes, prevention may encompass the slowing, arrest, delay, or other inhibition of diabetic progression, for example, from a non-overt diabetic status to diabetic status. Overt diabetes is characterized by known diagnostic factors, including, for example, elevated fasting blood glucose concentration (e.g. greater than 126 mg/dl when fasting) and other classical symptoms such as insufficient insulin production, hyperglycemia, diabetic ketoacidosis, and other symptoms of diabetes.

Certain methods of the invention will refer to increasing or decreasing a selected process or factor. References to “increasing” made herein, for example, increasing insulin release, may be with reference to any time scale, for example at scales of minutes, hours, days, or weeks, and may refer to specific activity at a selected time scale or an average activity over a selected time interval. For example, certain therapeutic effects may require days or weeks of treatment for efficacy to be assessed. Reference to increasing made herein, may refer to any comparative increase, such as an increase in the same subject at one time point vs. an earlier time point, or an increase observed in a subject relative to like untreated subjects. Similarly, reference to decreasing made herein may refer to any comparative decrease, such as a decrease in the same subject at one time point vs. an earlier time point, or a decrease observed in a subject relative to like untreated subjects. In various embodiments, the “increasing” (e.g., increasing GLP1R abundance and/or activity, increasing GLP1 activity) is, is about, or is at least about 1%, 5%, 10%, 15%, 20%, 50%, 70%, 100% or more relative to an increase in the same subject at one time point vs. an earlier time point, or an increase observed in a subject relative to like untreated subjects.

Administration. The methods of the invention encompass the administration of miR-192 agents to subjects. The administration may be any route, for example, systemic, for example comprising intravenous, intraperitoneal, subcutaneous, or oral administration. In one implementation the administration is local, for example, to the pancreas, pancreatic ducts, or other selected compartment of the body.

Administration of the miR-192 agent will be in a therapeutically effective amount. As used herein, “a therapeutically effective amount” means an amount sufficient to induce a measurable biological and/or therapeutic effect. In exemplary implementations, the biological effect may be any of: increasing insulin release, improving GSIS, improving glycemic control, increasing GLP1 activity, potentiating the effects of a GLP-1 agonist or DPP-IV inhibitor, or increasing the abundance of GLP1R in islet beta cells.

Doses administered may be calculated according to factors known in the art for determination of dosing regimens. Doses may be determined taking into account absorption, distribution, metabolism, elimination, toxicity, drug potency, patient need, and other factors which influence the selection of safe and efficacious doses.

Dosing regimen will depend on the nature of the miR-192 agent. For example, dosing may be multiple times per day, for example, with meals, or may be daily, multiple times weekly, monthly, or at any other selected interval commensurate with the effects of the agent.

Subjects. The methods of the invention encompass administration of miR-192 agents to subject. As used herein, a “subject” may be any animal species. The subject may be a human or a non-human animal such as a test animal or veterinary subject. Exemplary animals include human beings, non-human primates, cats, dogs, mice, rats, cows, pigs, horses and others. As used herein, a “diabetic subject” may be a subject having diabetes, or at risk of having diabetes, for example, a subject putatively having diabetes, or a subject diagnosed with diabetes.

In one implementation, the subject is a statin user. As used herein, a statin user is a subject that is or has been administered statins. Statins, as used herein, refers to any HMG-CoA reductase inhibitors known in the art. Exemplary statins include, for example, atorvastatin (sold as Lipitor), fluvastatin (sold as Lescol), pravastatin (sold as Lipostat), rosuvastatin (sold as Crestor), simvastatin (sold as Zocor), lovastatin (sold as Altoprev), and pitavastatin (sold as Livalo). A subject taking statins may be a subject that has been chronically administered statins in the past, is currently administered statins, or is a new user commencing statin administration.

Methods. In a first aspect, the scope of the invention is directed to a method of promoting GSIS in the beta cells of a subject, by administering to the subject a therapeutically effective amount of an miR-192 agent. “GSIS,” glucose-stimulated insulin release refers to a normal function of islet beta cells in the pancreas. A treatment which promotes GSIS refers to a treatment that achieve any one or more of the following therapeutic outcomes: increases insulin release in response to eating or to increased blood glucose levels; increases insulin release in response to GLP-1; increases insulin release in response to administration of an GLP-1 agonist or DPP-IV inhibitor; and maintains normal GSIS response in conjunction with the subject's use of statins.

In a second aspect, the scope of the invention is directed to the prevention and/or treatment of a condition. In one embodiment, the condition is diabetes. In one embodiment, the condition is Type II diabetes. In one embodiment, the condition is NOD, new onset diabetes. In one embodiment, the condition is NOD associated with statin use and the subject is a statin user. In one embodiment, the condition is hyperglycemia.

In some aspects, the disclosure provides methods of achieving weight loss in a subject by administration to the subject of a therapeutically effective amount of an miR-192 agent. In some embodiments, the subject is diagnosed as obese.

In some aspects, the disclosure provides methods of treating or preventing a cardiovascular disease in a subject by administration to the subject of a therapeutically effective amount of an miR-192 agent. In some embodiments, the cardiovascular disease is atherosclerotic cardiovascular disease.

Combination therapy. In one embodiment, the scope of the invention is directed to a method of potentiating a secondary therapeutic agent. As used herein, “potentiating” means any augmentation or enhancement of the effects of the enumerated secondary agent, for example, improving the activity of the agent, improving the therapeutic effect of the agent, reducing the amount of agent required to achieve a selected therapeutic effect; or any other increase in the potency of the selected agent. In a related implementation, the scope of the invention encompasses a method of treating or preventing a condition by the administration of an miR-192 agent and a secondary agent. In one embodiment, the condition is diabetes. In one embodiment, the condition is Type II diabetes. In one embodiment, the condition is NOD, new onset diabetes. In one embodiment, the condition is NOD associated with statin use and the subject is a statin user. In one embodiment, the condition is hyperglycemia.

Secondary therapeutic agents may include any drug or other therapeutic composition of matter. In a primary implementation, the secondary therapy is a GLP1 agonist or a DPP-IV inhibitor. Exemplary GLP1 agonists include, for example, exenatide, liraglutide, exentide dulaglutide. albiglutide, and semaglutide. Exemplary DPP-IV inhibitors include sitagliptin, metformin, saxagliptin, linagliptin, alogliptin, empagliflozin and vildagliptin.

In the combination therapy methods of the invention, the timing of the administration of the first, miR agent and the secondary treatment may be determined by one of skill in the art. In various implementations, the miR-192 treatment and secondary treatment(s) are administered any of contemporaneously, sequentially, or in an alternating sequence. In one embodiment, the first and second treatments are applied contemporaneously, i.e. simultaneously or overlapping in time. In one embodiment, the miR-192 treatment and secondary treatment(s) are administered in a pharmaceutical composition comprising a combination product, comprising a miR-192 agent and the secondary, for example, administered in a single dosage form.

### Part III. Diagnostic Methods

As demonstrated herein, certain subjects appear to be susceptible to statin induced NOD and such susceptibility corelates with statin-induced changes in miR-192 expression or abundance. Thus, miR-192 measurements in response to statins can be used to identify subjects at increased risk of developing NOD as result of statin use. In one implementation, the scope of the invention encompasses methods of assessing an enhanced risk of statin-induced NOD in a subject. By this method, non-diabetic subjects taking statins or prospectively taking statins can be assessed for an increased likelihood or probability of developing NOD as a result of statin use. In one embodiment, the subject is a subject that is currently using (i.e. is administered) statins. In another embodiment, the subject is a subject that is indicated for use of statins, for example, a subject with elevated cholesterol, but has not commenced statin use. By the methods, the subject's miR-192 response to statin administration is measured, wherein reduced miR-192 family member abundance indicates that the subject is susceptible to statin-induced repression of GSIS.

In Vivo Assay. In one implementation, the method comprises an in vivo assay wherein miR-192 response to statin administration in the subject is assessed. The general method comprises the following steps:
a first sample is obtained from a subject that is not using statins;
miR-192 family member abundance in the sample is measured;
a second sample is obtained from the subject after they have been administered statins;
miR-192 family member abundance in the sample measured;
the first and second measurements of miR-192 family member abundance are compared; and
risk of statin-induced NOD is assessed, wherein a decrease in miR-192 family member abundance after statin use is indicative of elevated risk of statin-induced NOD.

In the general method, the subject may be a subject that has not commenced statin use or who has not used statins for a long period of time, for example, weeks to months. In another implementation, the subject is a subject that has previously used statins and is pausing statin use for purposes of the test, for example, having abstained from statin use for one, two, three, four, or five days, a period of one, two, or three weeks, etc.

In one implementation of the method of the invention, the subject may be a fasted subject, i.e., a subject that has not eaten for a period of hours, for example, eight hours, ten hours, overnight, or other period of time such that food intake and metabolism does not affect miR-192 family member abundance. In another implementation of the method of the invention, the subject is a fed subject, for example, a subject that has just eaten a meal.

In the method of the invention, the sample may comprise any sample indicative of miR-192 family member activity or abundance in the B cells of the subject. Such activity or abundance is correlated with circulating levels of miR-192 family members in the blood. Accordingly, the sample may be a blood sample, or serum derived therefrom. In other embodiments, the sample is urine, saliva, or sweat.

In the method of the invention, the abundance of one or more miR-192 family members is assessed. In a primary embodiment, the miR-192 family member is miR-192, for example, miR-192-5p. In other implementations, the one or more miR-192 family members may comprise miR-192-3p, miR-194, or miR-215.

Abundance of the target species may be assessed by any suitable measurement method for quantifying micro-RNAs as known in the art. Exemplary methods include real-time reverse transcription PCR (qPCR), microarray analysis, sequencing, or other probe-based detection methods. Abundance may be assessed as a concentration value, copy number, or other measures of micro-RNA abundance.

Comparison of pre-statin and post-statin miR-192 family member abundance may be achieved by a simple comparison of two measurements. In an alternative implementation, multiple measurements are taken both pre- and post-statin use, in order to increase signal to noise of the assay, or to monitor the time course of any changes in miR-192 family member abundance as a result of statin administration. “Decreased” miR-192 family member abundance may be any biologically relevant reduction in the abundance of the selected miR-192 moiety measured in the sample, indicative of statin-induced inhibition of miR-192 production or circulation. In various embodiments, “decreased” status may be designated for a reduction of at least 5%, at least 7.5%, at least 10%, at least 15%, at least 20%, at least 25%, at least 30%, at least 35%, at least 40%, at least 45%, at least 50% or greater reduction.

The method of the invention may comprise additional steps to be performed upon determination that the subject is at elevated risk of statin-induced NOD. Steps may include, for example, taking the subject off of statins, not commencing statin use, changing therapeutic agents to a different statin or other agent, and initiating periodic monitoring of miR-192, blood glucose, or other measures of diabetic risk. In some embodiments, a glucose-lowering regimen is initiated upon determination that the subject is at elevated risk of statin-induced NOD. In some embodiments, the glucose-lowering regimen comprises administration of insulin, metformin, sulfonylureas, sodium-glucose co-transporter inhibitors, thiazolidinediones, dipeptidyl peptidase-4 (DPP-4) inhibitors, pramlintide, alpha-glucosidase inhibitors, or a combination thereof to the subject determined to be at elevated risk of statin-induced NOD. In another embodiment, miR-192 data is incorporated into a diagnostic panel for assessing statin risk, for example assessment of miR-192 can be performed in combination with assessment of SLCO1B1 polymorphisms in the subject.

In Vitro Assay. In another implementation, the scope of the invention encompasses methods of assessing an enhanced risk of statin-induced NOD in a subject by use of cells derived from the subject. As demonstrated herein, the response of cultured cells derived from subjects is correlated to miR-192 biology in the body, and thus, cells derived from a subject may serve as a proxy by which statin effects on glucose metabolism can be assessed. By this method, non-diabetic subjects taking statins or prospectively taking statins can be assessed for an increased likelihood or probability of experiencing NOD as a result of statin use. In one embodiment, the subject is a subject that is currently using (i.e. is administered) statins. In another embodiment, the subject is a subject that is indicated for use of statins, for example, a subject with elevated cholesterol, but has not commenced statin use.

In a general implementation, the method encompasses the following steps:
a cellular sample is obtained from a subject;
a cell culture derived from the sample is established;
miR-192 family member abundance in the cells is measured in the absence of statins;
one or more selected statins are applied to the cell culture;
miR-192 family member abundance in the cells is measured after the application of statins;
the first and second measurements of miR-192 family member abundance are compared; and
risk of statin-induced NOD is assessed,
wherein a decrease in miR-192 family member abundance in response to statin use is indicative of elevated risk of statin-induced NOD.

In this in vitro assay, the cellular sample and cell culture derived therefrom may comprise any biologically relevant cell types. For example, in one embodiment, an induced pluripotent stem cell culture is established from a sample comprising blood. For example, in one embodiment the iPSCs are reprogrammed from CD34⁺ peripheral blood mononuclear cells (PBMCs) isolated from blood samples the subject, for example, as described in the Examples section herein.

As in the in vivo assay, the subject may be a subject that is taking statins or is potentially commencing statin use. As in the in vivo assay, one or more miR-192 family members may be selected for measurement, for example, miR-192-5p. As in the in vivo assay, the measurement may be achieved by any methodology known in the art, for example qPCR or microarray analysis. A “decreased” miR-192 abundance may be any biologically relevant decrease, for example a reduction of at least 5%, at least 7.5%, at least 10%, at least 15%, at least 20%, at least 25%, at least 30%, at least 35%, at least 40%, at least 45%, at least 50% or greater reduction.

If the assay indicated statin-induced decrease of miR-192 abundance in the cells, the subject is deemed to have an elevated risk of developing statin-induced NOD. In such case, the method of the invention may comprise the further step of performing a selected intervention, for example, discontinuing statin use by the subject, not commencing statin use, switching the subject to a different therapeutic agent for their condition (e.g. high cholesterol), or initiating a monitoring program for miR-192 changes or diabetic risk factors, as known in the art, for example A1C or blood glucose testing at regular intervals to detect indicia of diabetes. In one embodiment, the selected intervention is the co-administration of both statins and an miR-192 agent and/or a GLP-1 agonist in order to prevent or reduce the risk of the subject having statin-induced NOD.

Kits. The scope of the invention further encompasses diagnostic kits for performance of the aforementioned assays. The kits of the invention will comprise various physical elements for performance of a diagnostic assay of the invention, for example, the foregoing in vivo and in vitro assays, wherein the elements are packaged in a common container, e.g. a box, or otherwise collected to facilitate performance of the methods of the invention. For example, in one embodiment, the kit comprises a qPCR kit for measurement of one or more selected miR-192 family members, for example, miR-192-5p. Exemplary elements of the kit may include reverse transcriptase, miR-192 family member sequence standards for assay calibration, primers such as hairpin loop primers for RT-amplification and PCR amplification, thermal cycling tubes, and software for data analysis, miRNA quantification and output of results. In another embodiment, the kit comprises a microarray assay for quantification of the selected miR-192 species, for example comprising elements such as reverse transcriptase, labeling agents such as fluorophores or fluorescent proteins, immobilized probes that selectively bind to the target micro-RNAs (such as complementary sequences), target micro-RNA standards for assay calibration, and software for data collection, analysis, and output. In one embodiment, the diagnostic kit of the invention comprises a quantity of probes, i.e. sequences complementary to one or more miR-192 family members capable of binding or capturing (for example, on a solid and/or labeled substrate) miR-192 family members in a sample. In one embodiment, the probes are labeled (for example, the label comprising any detectable moiety, e.g., a fluorophore, fluorescent protein) or functionalized for labeling (i.e. comprising a chemical conjugation handle or moiety for attaching a detectable label).

In one embodiment, the diagnostic kit of the invention comprises one or more detection agents for quantifying miR-192 and one or more additional detection agents for quantifying another biomarker of risk for statin-induced NOD, for example, detection agents for quantifying or assessing SCLO1B polymorphisms.

### EXAMPLES

### Example 1

### MIR-192 is a Novel Regulator of GLP1R and Insulin Secretion and Contributes to Statin-Induced Diabetes

### 1. Introduction

Statins, a class of HMG-COA reductase inhibitors, are the most widely prescribed drug for the prevention of cardiovascular disease in the US, with ~56 million statin eligible individuals based on the 2018 American College of Cardiology-American Heart Association Guidelines for cholesterol management¹. Recent clinical trial reports indicate that statins elevate the probability of developing new-onset diabetes (NOD)^{2,3}, prompting the Food and Drug Administration to add information to statin labels regarding this increased risk. In both randomized controlled clinical trials and observational studies, the incidence of NOD in statin users has been recently reported to be greater than 9-12%², and this figure has been found to be even higher (~30% or more) in women³. While it has been shown that the new-onset of diabetes does not outweigh the benefits of statins on cardiovascular disease risk⁴, these observations signify the potential for other long-term risks in a relatively high proportion of statin users, and highlight the need to elucidate the underlying mechanism(s) responsible for statin-induced NOD.

Glucagon-like peptide-1 (GLP-1) released from enteroendocrine L cells exhibit its incretin-like activity in response to a meal through its receptor GLP1R in β-cells, augment glucose-stimulated insulin secretion (GSIS)⁵. In addition to potentiating GSIS, GLP1R signaling also promotes β-cell proliferation and insulin biosynthesis in the pancreas, and reduces glucose production, appetite and gastric emptying in peripheral tissues⁶. Recently developed GLP1R agonists like exenatide and liraglutide have proven to be incredibly efficacious drugs for individuals with diabetes, both in terms of managing blood glucose levels and preventing cardiovascular disease and death, a well-established long-term adverse outcome of type 2 diabetes mellitus (T2DM). The remarkable success of GLP1R agonists in the management and treatment of T2DM^{7,8} demonstrates the potential clinical significance of identifying molecular regulator of GLP1R.

Multiple independent studies in β-cell lines and in human and mouse islet cells 9-11 have demonstrated that statins impair both endogenous and GLP-1 mediated insulin secretion. In murine islet cells, this reduction is rapid, reversible, and occurs even in high-glucose and without effecting Ca²⁺ concentrations⁹. Although this effect is well documented, its molecular basis is not clear. While some studies have suggested a role for statin inhibition of isoprenoid synthesis¹¹ or mitochondrial function¹⁰, others have refuted these theories⁹. Hypothesizing that statins can be used as tools to expose molecular mechanisms of β-cell dysfunction, we created a repository of induced pluripotent stem cells (iPSCs) from statin users who developed NOD (NOD Cases) or maintained normal fasting glucose after statin initiation (Controls). Through RNAseq analysis, we identified MIR-194-2 host gene (MIR-194-2HG) as one of the most differentially expressed genes between iPSCs from NOD Cases vs. Controls after in vitro statin exposure.

MIR-194-2HG is a long non-coding RNA that encompasses two miRNAs, MIR-192 and MIR-194. Circulating MIR-192-5p levels are reportedly higher in individuals with impaired fasting glucose and patients with either type 1 or type 2 diabetes compared to those with normal fasting glucose^{12,13}. However, studies are limited and the relationship between circulating MIR-192-5p abundance and diabetes is unclear and there is a need in the art for determination of the underlying processes that mediate this unexplained phenomenon.

Here, we provide evidence that MIR-192-5p impacts GSIS through indirect regulation of GLP1R mediated by the GLP1R 3′ UTR. In addition, MIR-192-5p reverses statin-induced impairment in insulin secretion. These findings provide the art with 1) an understanding of the pathobiology of the development of diabetes, 2) specific molecular processes that underlie risk of developing T2DM in statin users, and 3) T2DM management given the key role of GLP1R activation in the efficacy of GLP1R agonists.

### 2. Materials and Methods

### 2.1. Description of the Kaiser Permanente of Northern California Population (KPNC)

Kaiser Permanente of Northern California (KPNC) is one of the largest group practice-based integrated healthcare delivery systems in the US. KPNC provides comprehensive medical services to over 3 million members through 20 different medical centers to 3.3 million members in a 14-county region that includes the San Francisco Bay and Sacramento metropolitan areas, employing >7,000 physicians. KPNC has well-developed guidelines and standards of care that are uniformly adopted across all sites. In the geographic areas served by Kaiser, approximately 33% of the general population has KPNC coverage. Sociodemographic characteristics are generally representative of the underlying population, except for an under-representation of the extremes of income. For example, the KPNC vs. Bay Area Metropolitan Statistical Area ethnic composition is as follows: white (66% vs. 58%), black (6% vs. 8%), Asian (16% vs. 19%), and other (12% vs. 15%). 19% of the Bay Area Metropolitan Statistical Area self-identifies as Hispanic/Latino, compared to 11% of KPNC members. The 5-year (2009-2013) membership retention rates of persons over ages 40 and 66 are about 65 and 80%, respectively.

### 2.2. KPNC Electronic Databases

Each Kaiser member is assigned a lifetime unique identifier that is used throughout all databases. Information was drawn from one of three databases:

KP HealthConnect® is a fully integrated electronic health record that documents ambulatory visit check-in, hospital-based utilization, clinical documentation of diagnoses, orders for tests and procedures, and prescribed medications. Other functions include documentation of labs, radiology, oncology, and other diagnostic testing, as well as tracking of outside claims and referrals.

Pharmacy Information Management System contains prescription medications dispensed at KPNC hospitals, medical centers, and medical offices for either inpatient or outpatient use. Implemented in all facilities in 1994, the database contains, but is not limited to, medical record number, cost, prescribing practitioner, medicine name, National Drug Code, date of prescription, date medication was dispensed, and dosage and refill information. This system is used to generate labels for dispensing drugs, thus data are not only collected in real time, but the information contained within this system is considered extremely accurate.

Laboratory Utilization and Reporting System captures all ordered and performed laboratory tests from KPNC hospitals, medical centers, and medical offices. The database was created in 1994 and contains, but is not limited to, medical record number, facility code, name of ordering provider, test or procedure name, results, date of test/procedure/result, and abnormal or out-of-range flags.

### 2.3. Identification of Statin Users with (NOD Cohort) or without (Control Cohort) New-Onset Diabetes

Using the pharmacy records, we identified individuals who had his/her first statin (simvastatin, lovastatin, atorvastatin, pravastatin, rosuvastatin, cerivastatin, or pitavastatin) prescription between the ages of 40-75, and documented continuous statin use for 3 years. Continuous statin use was defined as having greater than 8 30-day prescription refills per year or greater than 3 90-day prescription refills per year. Individuals prescribed statin combinations, such as Advicor (niacin/lovastatin) or Vytorin (ezetimibe/simvastatin) were excluded.

Individuals with evidence of diabetes prior to the date of the first statin prescription or within the first 3 months after the start of statin use were excluded. Evidence of diabetes included either i) diabetes diagnosis based on ICD9 codes for type I, II or gestation diabetes or ii) prescription for glucose lowering drugs such as oral hypoglycemics (alpha-blucosidase inhibitors, dipeptidyl peptidase-4 inhibitors, meglitinides, sulfonylureas), anti-hyperglycemic agents (biguanides, thiazolidinediones, dipeptidyl peptidase-4 inhibitors, meglitinides, sulfonylureas), and insulins. In addition, individuals with prescriptions for glucose raising drugs, such as oral corticosteroids or ICD9/CPT codes for bariatric surgery, in the 3 years prior to start of statin treatment, through the 3 years after the start of statin treatment were excluded.

To ensure that individuals were not diabetic prior to the start of statin treatment, individuals were required to have at least two fasting glucose measures which must all be between 50-110 mg/dl and within 30 units of each other. Individuals with outpatient fasting glucose measure greater than or equal to 126 mg/dL within the first 3 months after the start of statin treatment were excluded. FG values <30 mg/dL or >600 mg/dl were excluded (as these are likely attributable to laboratory error). Individual who developed NOD in the 3 years after statin initiation were identified and recruited with diabetes defined as: 1) FG 126 mg/dl, 2) diabetes ICD9 code, and/or 3) prescription of a glucose lowering drug.

Controls were defined as statin users with normal glycemia (all FG<110 mg/dL) prior to statin initiation and during the first 5 years on-treatment. Individuals with a diabetes ICD code or use of glucose-modifying drug were excluded.

Sodium palmitate (#P9767), sodium oleate (#07501), arachidonic acid sodium salt (#SML1395), and docosahexaenoic acid sodium salt (#D8768) were purchased from Sigma-Aldrich.

### 2.5. Creation and Authentication of iPSCs

iPSCs were reprogrammed from CD34⁺ peripheral blood mononuclear cells (PBMCs) isolated from blood samples of study participants and validated as previously described¹⁴. Briefly, CD34⁺ PBMCs were subject to expansion, nucleofected with episomal vectors of POU5F1, SOX2, KLF4, L-MYC, LIN28, EBNA1, and shRNA for TP53¹⁵, and seeded onto mitomycin C treated SNL feeder cells. After emergence of iPSC colonies, TRA-1-60 expressing cells were selected using Magnetic Activated Cell Sorting to obtain one pooled cultured iPSC line per study participant. Expression levels of pluripotency markers POU5F1 and TRA-1-60, and differentiation marker SSEA-1 in iPSCs were visualized with immunohistochemistry and quantified with flow cytometry. Representative lines were sent for KaryoStat, PluriTest (Thermo Fisher Scientific), or karyotype (Cedar Sinai RMI iPSC Core) analyses. The pluripotency of iPSCs was further confirmed by positive differentiation of representative lines into endoderm, mesoderm and ectoderm using the STEMdiff Trilineage Differentiation Kit (StemCell Technologies, #05230) followed by immunostaining and flow cytometry analyses.

### 2.6. Cell Culture

INS-1 rat insulinoma cell line was obtained from UC Berkeley Cell Culture Facility and maintained in RPMI 1640 media with 10% fetal bovine serum (FBS), 1 mM sodium pyruvate, 10 mM HEPES, and 55 μM 2-Mercaptoethanol. iPSCs were cultured on plates coated with Cultrex reduced growth factor basement membrane (Trevigen #3533-001-02), fed daily with mTeSR1 (StemCell Technologies #85850), and passaged routinely using ReLeSR (StemCell Technologies #05872). All cell lines were kept at 37 °C in a humidified incubator containing 5% CO₂.

### 2.7. Transfection of miRNA Mimic and Inhibitor

INS-1 cells were transfected with miRVana miRNA mimic (Invitrogen, #4464066, Assay ID MC10456) or inhibitor (Invitrogen, #4464084, Assay ID MH10456) for has-miR-192-5p, or equal concentration of negative controls of miRVana miRNA mimic (Invitrogen, #4464058) or inhibitor (Invitrogen, #4464076) using Lipofectamine RNAiMAX (Invitrogen, #13778075).

### 2.8. RNA Isolation and qPCR

Total RNA was isolated from cells with miRVana miRNA isolation kit (Thermo Fisher Scientific, #AM1560) 48 hours after transfection. The synthetic cel-miR-39-3p (Qiagen, #MSY0000010) was added to cell conditioned media or human plasma samples as spike-in control, and total RNA was isolated from media or human plasma with miRVana PARIS RNA and native protein purification kit (Thermo Fisher Scientific, #AM1556). The cDNA was prepared from 10 ng of total RNA from cells, or 2 μl of total RNA from media, with TaqMan advanced miRNA cDNA synthesis kit (Thermo Fisher Scientific, #A28007). Real-time PCR with TaqMan advanced miRNA assays (Thermo Fisher Scientific, #A25576) was used to quantify mature miRNA in the samples: has-miR-192-5p (478262_miR), hsa-miR-16-5p (477860_miR), or cel-miR-39-3p (478293_miR).

### 2.9. Western Blots

Protein extracts were prepared using CelLytic M buffer (Sigma-Aldrich, #C2978) with protease inhibitor cocktail (Thermo Fisher Scientific, #78429), and protein concentration was determined with a Bradford based protein assay (Bio-Rad, #5000002). Equal amount of protein samples was separated by electrophoresis on a 4% to 20% Tris-Glycine gel (Thermo Fisher Scientific, #XP04202BOX) and transferred to a PVDF membrane, which was then fixed with 0.4% paraformaldehyde for 15 minutes. Antigen retrieval was performed with 10 mM citric acid buffer at pH 6.0 at 95 °C for 5 minutes (reference) prior to immunoblotting. Antibodies used in the current study include anti-GLP1R (Iowa DSHB, #Mab 7F38) and anti-GAPDH (Santa Cruz Biotechnology, #sc-166545).

### 2.10. 3′ UTR Reporter Constructs

The LightSwitch luciferase reporter construct, pLS-GLP1R-3′UTR, was purchased from Active Motif (#S810047), and encompasses bases 1411-3187 of the GLP1R transcript NM_002062.5. The following pairs of primers were used to mutate the predicted miR-192 binding site AGGTCAA at NM_002062.5 position 1813-1819 to GTAGTGC: 5′-GTGCCGGCTTATTAGTGAAACTGGGGCTTG-3′ (SEQ ID NO: 6) and 5′-TACTGAGTTTGAGTCTGGGGTTGATTTGCGGC-3′ (SEQ ID NO: 7). Empty (SwitchGear Genomics, #S890005) or 3′ UTR of housekeeping gene GAPDH (SwitchGear Genomics, #S801378) vectors were used as non-targeting control constructs in luciferase reporter assays. SCD 3′ UTR reporter construct was a kind gift from Dr. Vesa M. Olkkonen.

### 2.11. Luciferase Reporter Assay

Cells were plated in white 96-well plates overnight and co-transfected for 24 hours with each luciferase reporter construct and miR-192 mimic, inhibitor, or non-targeting negative controls using DharmaFECT DUO transfection reagent (Dharmacon, #T-2010-02) following manufacture's instruction. Luciferase activity was measured with LightSwitch assay reagents (SwitchGear Genomics, #LS100) on a Synergy H1 microplate reader.

### 2.12. Insulin Secretion Assay

INS-1 cells were plated in 24-well plates and transfected the next day with various miRNA for 48 hours. On the day of insulin secretion assay, transfected INS-1 cells were pre-incubated in KREBS-Ringer Bicarbonate (KRB) buffer containing 1% BSA for 30 min at 37 °C, and then incubated with 2.8 or 17.8 mM glucose and 50 nM GLP-1 in KRB buffer containing 1% BSA for 30 min. The insulin containing buffer was then collected from each well and centrifuged at 450×g for 5 min. The resulting supernatant was stored at −20 °C until ready for insulin measurement, whereas the cells were lysed in CelLytic M for protein quantification using a Bradford protein assay. Insulin concentrations were measured using a rat/mouse insulin ELISA kit (Millipore, #EZRMI-13K) following the manufacturer's instructions.

### 3. Results

### Generation of a Cohort of New Onset Diabetes (NOD) Cases and Controls

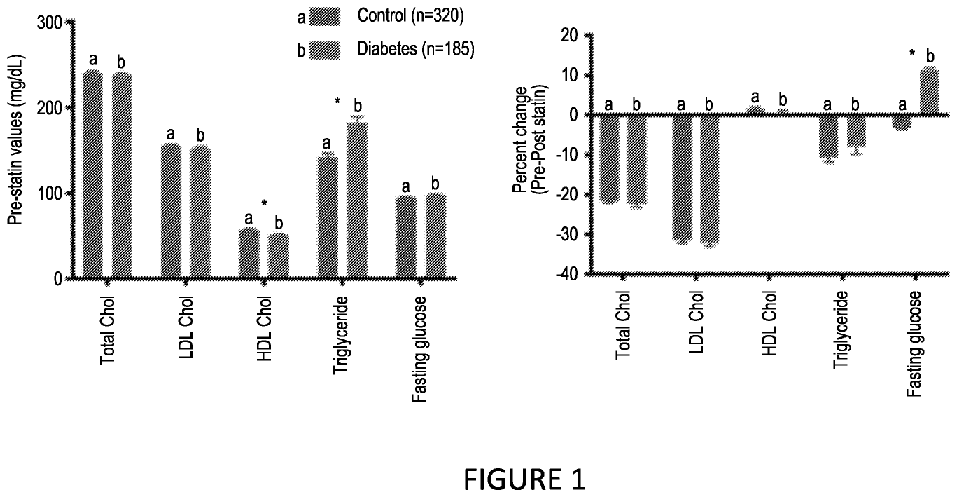

Using the electronic health records of Kaiser Permanente of Northern California, we identified and recruited 185 cases of NOD in statin users (NOD cases) and 320 statin users who maintained normal glycemia (controls). NOD Cases were defined as statin users with normal glycemia prior to initiation and who had either evidence of diabetes by a fasting glucose (FG) measure greater than or equal to 126 mg/dL, use of a glucose lowering drug and/or a diabetes ICD code, in the first 3 years on-treatment documented by continuous prescription refills. In addition, individuals with very large increases in FG after statin initiation were also recruited. Individuals with any diabetes ICD code, use of a glucose-lowering or raising drug, FG greater than or equal to 126 mg/dL, or HbA1c greater than or equal to 6.5% prior to statin initiation were excluded. Controls were defined as statin users with normal glycemia (all FG<110 mg/dL) prior to statin initiation and during the first 5 years on-treatment. Individuals with a diabetes ICD code or use of glucose-modifying drug were excluded. Recruited individuals were ~50% women, and self-identified as White, Asian, Black or Hispanic. Pre-statin plasma FG measures and the statin induced change in LDLC were identical between the NOD cases and controls (FIG. 1).

### Generation of iPSCs from NOD Cases Vs. Controls

Patient-derived iPSCs retain the genetic characteristics of the donor, exhibit self-renewal, and can be cryopreserved, rendering them to be a highly versatile cellular model^{16,17}. Peripheral blood mononuclear cells (PBMCs) were collected from 24 NOD cases and 24 controls, and reprogrammed into iPSC lines as we previously described¹⁴. Immunohistochemistry and flow cytometry were used to verify iPSC pluripotency marker expression¹⁴. Of the cells within an individual line, 91.7% (range 66.4-98.9%) were positive for TRA-1-60 expression, 93.1% (range 73.2-98.9%) were positive for POU5F1, and 2.8% (range 0.2-4.8%) were positive for SSEA-1. All lines were greater than 90% positive for TRA-1-60 and/or POU5F1, and less than 5% positive for SSEA-1.

### Identification of MIR-192 and MIR-194 as Putative Factors Underlying NOD

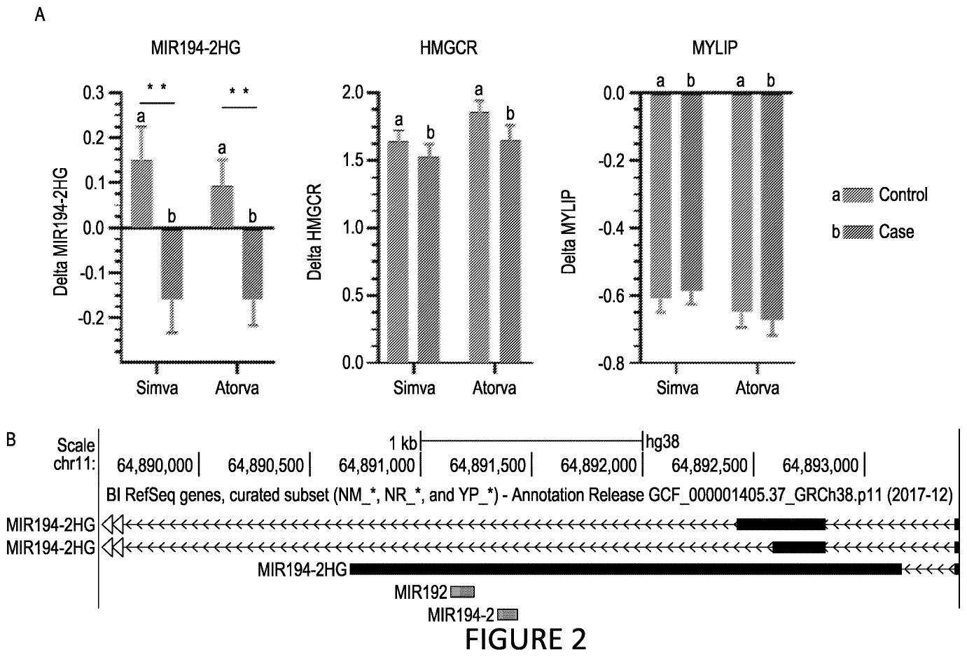

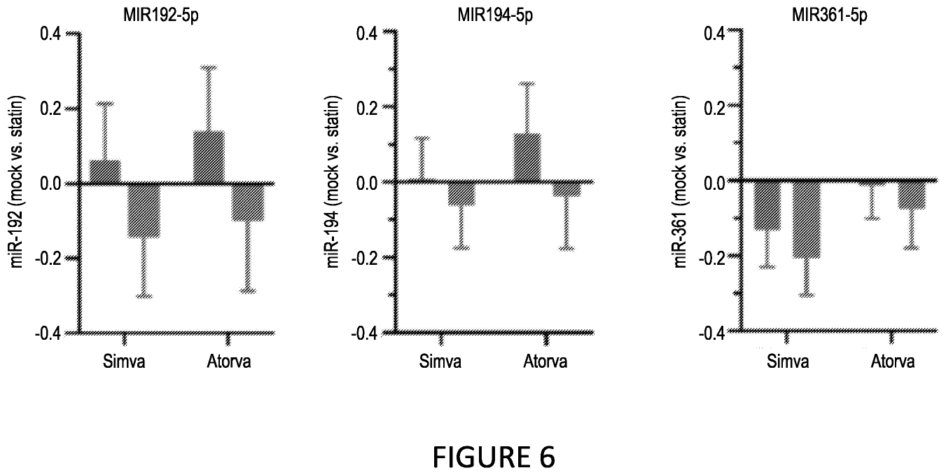

We incubated iPSCs from NOD cases vs. controls (n=24/group) with 250 nM atorvastatin, 500 nM simvastatin or a mock buffer for 24 hours, after which RNAseq was performed on polyA-isolated strand-specific paired-end libraries as we described¹⁸. These statin concentrations were identified using dose-response analyses in 6 iPSC lines as the lowest concentration that generated a robust and reproducible increase in HMGCR and decrease in MYLIP, well-known effects of statin treatment^{19,20}. Differences in the variance stabilized levels of statin vs. mock-treated transcripts between cases and controls were assessed via t-test, and a long non-coding RNA, MIR-194-2 host gene (MIR-194-2HG) was one of the most differentially expressed genes identified. While statin exposure increased MIR-194-2HG expression in the controls, both statin types reduced MIR-194-2HG in the NOD cases, p<0.01 (FIG. 2A). As the name suggests, MIR-194-2HG encodes two miRNAs, MIR-192 and MIR-194 (FIG. 2B), and not surprisingly, both statin types increased intracellular MIR-192-5p and MIR-194-5p in the controls, but decreased levels in the cases (FIG. 6). As positive controls, we quantified HMGCR and MYLIP to demonstrate the expected increased and decreased gene expression with statin treatment (FIG. 2A), and MIR361-5p as a negative control showing no difference in statin induced change between the NOD cases and controls (FIG. 6).

### MIR-192-5p Regulates GLP1R and Insulin Secretion

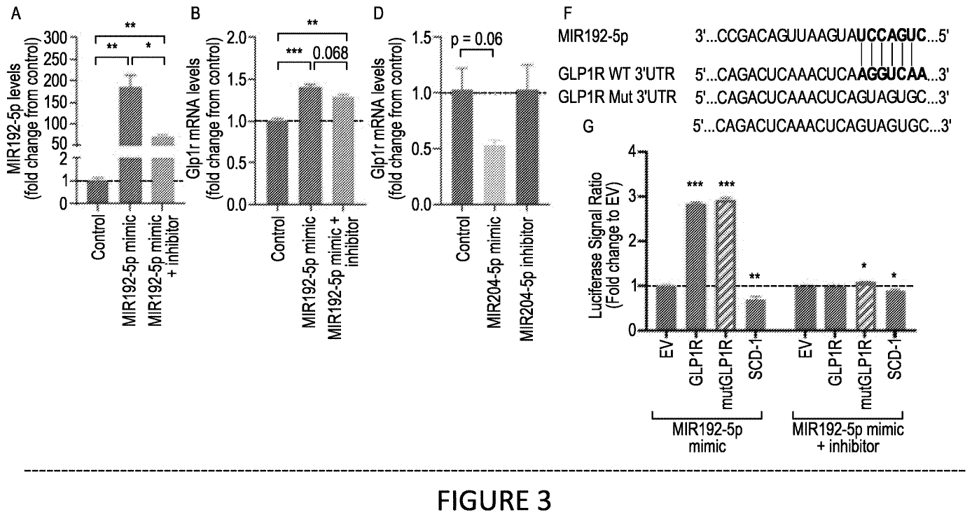

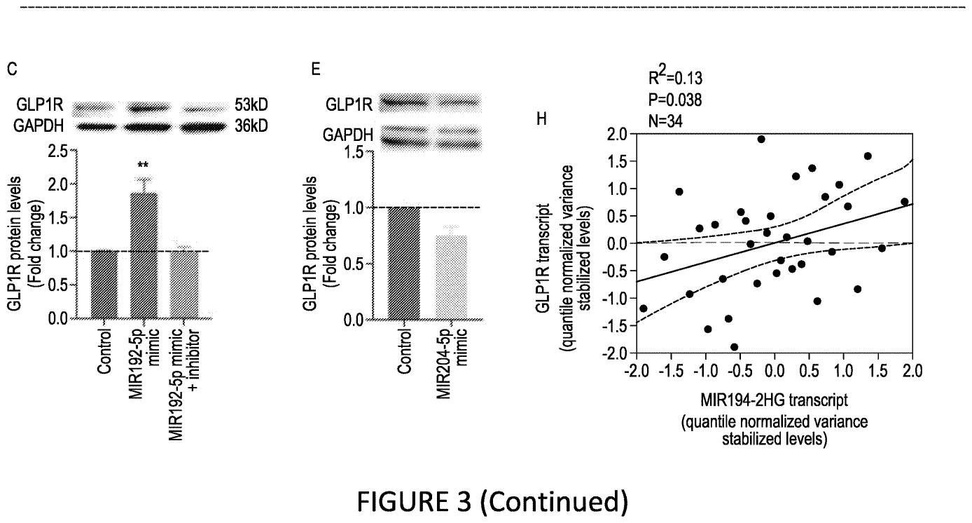

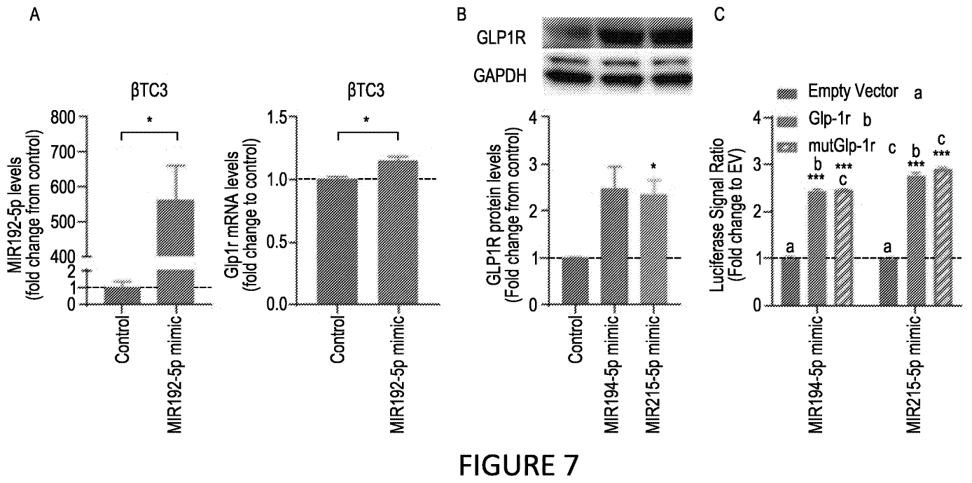

Elevated levels of circulating MIR-192-5p has been identified in individuals with either diabetes (type 1 or 2) or impaired fasting glucose compared to those with normal fasting glucose²¹⁻²³. Two prior reports have utilized miRNA target prediction tool TargetScan to identify GLP1R as a potential target of MIR-192-5p and shown that MIR-192-5p overexpression suppressed GLP1R expression in human renal tubular epithelial cell line HK-2²⁴, and human enteroendocrine cell line NCI-H716¹³. MIR-192 has tissue restricted expression, but is found in the pancreas²⁵. In fact, MIR-192-5p is among the top 10 most abundant miRNAs in islets and β-cells²⁶. To test whether MIR-192-5p regulates GLP1R in a similar fashion in β-cells, we transfected INS-1 cells, a rat pancreatic β-cells line, with a MIR-192-5p mimic. Glp1r transcript levels were upregulated by 1.41±0.03 fold (n=6, P<0.0001) and protein levels were upregulated by 1.87±0.20 fold (n=6, P<0.01) with miR-192 mimic, an effect that was reversed with addition of a MIR-192-5p inhibitor (FIG. 3A-C). Similar results on Glp1r transcript levels were seen in a mouse insulinoma cell line, BTC3 (FIG. 7A). Significant increases were also observed with a luciferase reporter containing human GLP1R 3′ UTR in INS-1s (1.54±0.18 fold, n=5, P<0.01) (FIG. 3G). However, increased luciferase signal remained despite disruption of the predicted MIR-192-5p binding site within the GLP1R 3′ UTR (2.06±0.24 fold, n=3, P<0.01). Since miRNAs typically reduce levels of target gene transcript and/or protein²⁷, we tested the effect of the MIR-192-5p mimic on a luciferase construct containing the 3′ UTR of SCD, a direct target of MIR-192-5p²⁸, and found the expected reduction in luciferase (FIG. 3G). Lastly, we observed a positive correlation between MIR-194-2HG and GLP1R transcript levels in our iPSCs, p=0.04, R2=0.13, N=34 (FIG. 3H), which together support the likelihood that more MIR-192-5p leads to increased GLP1R.

The unexpected finding that MIR-192-5p increases GLP1R, and the fact that its mimic increased luciferase levels even after disruption of the putative MIR-192-5p binding site, strongly suggests that MIR-192-5p up-regulates GLP1R through indirect mechanisms. Since MIR-192 and MIR-194 are created from the same primary transcript (MIR-194-2HG), FIG. 2B, we hypothesized that MIR-194-5p may have similar functional effects. Similarly, although MIR-215-5p is processed from an independent transcript from MIR-192 and 194, it has an identical seed sequence as MIR-192-5p and thus most likely targets the same genes as MIR-192-5p. We found that mimics for both MIR-194-5p and 215-5p lead to nearly identical increases in GLP1R protein levels (FIG. 7B), effects that were confirmed in the GLP1R 3′ UTR luciferase constructs containing both the endogenous and mutant forms (FIG. 7C). Notably, like MIR-192, both MIR-194 and 215 show tissue restricted expression that is predominantly in the pancreas, small intestine and colon²⁵. Together these findings show that MIR-192, 194 and 215 are a trio of miRNAs that are novel regulators of GLP1R that function through the GLP1R 3′ UTR, and that the relationship between MIR-192 and diabetes may be attributed to regulation of GLP1R and GLP-1 augmented glucose-stimulated insulin secretion (GSIS).

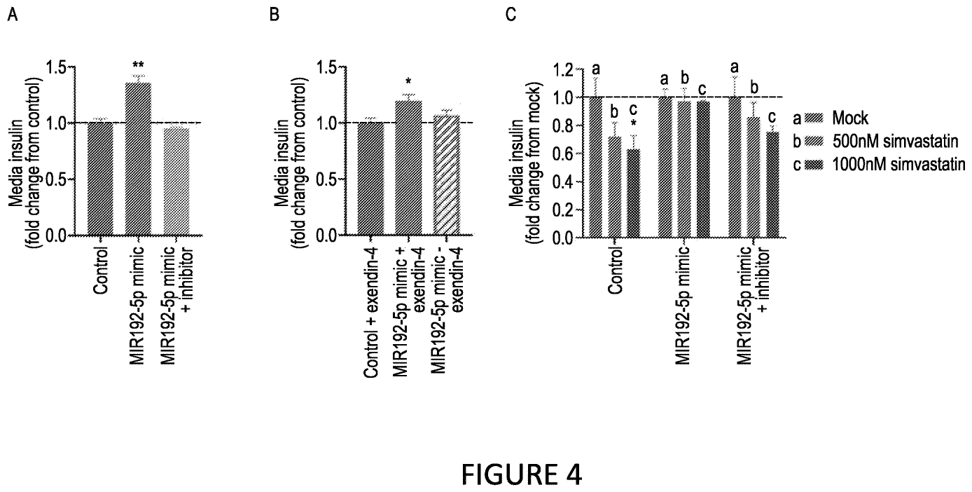

Consistent with our hypothesis, we found 35% greater insulin levels in MIR-192-5p mimic transfected INS-1 conditioned media after stimulation with 17.8 mM glucose+50 nM GLP-1, effects that were reversed with addition of the MIR-192-5p inhibitor (FIG. 4A). We also found that the MIR-192-5p mimic increased INS-1 media insulin levels when stimulated with 16 mM glucose+100 nM exendin-4, which is a GLP1R agonist. However, in the absence of exendin-4, MIR-192-5p mimic failed to increase media insulin (FIG. 4B), indicating that the effect of MIR-192-5p on media insulin is GLP1R dependent.

### MIR-192-5p Rescues Statin-Induced Impairment of Glucose-Stimulated Insulin Secretion

To explore the effects of statin on insulin secretion, we exposed INS-1 cells to increasing concentrations of simvastatin for 30 min under stimulatory condition of 17.8 mM glucose and 10 nM exendin-4 and found a reduction in media insulin levels (FIG. 4C), consistent with literature reports. The reduction in media insulin observed in simvastatin treated INS-1 cells was rescued by MIR-192-5p overexpression, while addition of the MIR-192-5p inhibitor reverses this effect, where statin treatment reduced media insulin levels (FIG. 4C). Together these findings strongly support our hypothesis that statin treatment impairs GSIS, and that this effect can be reversed by MIR-192-5p.

### 4. Discussion

Statins are a commonly prescribed CVD drug class known to impair insulin secretion, and there is extensive evidence that statin use accelerates the onset of T2DM. This risk is recognized by the American College of Cardiology, the FDA, and the European Medicines Agency 29, and has led to changes in statin use guidelines and warning labels on statin prescriptions. While a variety of mechanisms have been proposed to contribute to impaired glucose metabolism and risk for T2DM with statin treatment, the supporting evidence in humans is inconclusive. One of the major challenges is to identify statin treatment as the cause of NOD because 1) individuals who are often at high risk for cardiovascular disease are often those at high risk for metabolic disease (including T2DM), and 2) development of T2DM (irrespective of statin use) usually entails a gradual increase in plasma fasting glucose over the course of several years. Through electronic health records from the members of KPNC, we carefully identified a well-defined cohort of statin users to access statin effects on NOD. The similarities in lipid profiles between NOC cases and controls strongly suggests that the NOD cases have an inherently greater susceptibility to developing diabetes (and not greater statin exposure), a risk exacerbated by statin use.

Glycemic control in response to a meal is maintained through the secretion of GLP-1 and GIP (glucose-dependent insulinotropic polypeptide), incretin hormones secreted by the intestine that upon binding to their receptors (GLP1R and GIPR respectively) in pancreatic β-cells, augment GSIS³⁰. Under normal conditions, GIP is the major hormone responsible for enabling GSIS³¹. However, in the diabetic state, GIPR becomes insensitive, while GLP1R remains functional³². Two classes of agents commonly used to treat T2DM function through activation of GLP1R: 1) GLP1R agonists (e.g. exenatide, liraglutide), are GLP-1 mimics that induce a more robust increase in GSIS than GLP-1, which is subject to rapid enzymatic decay by dipeptidyl-peptidase-IV (DPP-IV). 2) DPP-IV inhibitors (e.g. sitagliptin, vildagliptin) stabilize GLP-1, and therefore their clinical benefit is also mediated by GLP1R³³. Importantly, GLP1R agonists have been shown to not only reduce blood glucose, but also to protect against atherosclerotic cardiovascular disease^{34,42}, the leading cause of death in patients with T2DM³⁵. The discovery of novel regulators of GLP1R could be used to inform the development of new therapeutics designed to augment the effects of GLP1R agonists or DPP-IV inhibitors.

Although the molecular basis of GLP1R signaling cascade has been extensively studied³⁶, considerably less is known regarding the regulation of GLP1R levels. At the protein level, GLP1R is known to undergo rapid homologous desensitization, leading to reduced GLP-1 binding capacity³⁷, while N-glycosylation has been shown to increase receptor half-life³⁸. In vitro studies have found that Glp1r transcript levels are modulated by high dose, but not low dose glucose³⁹, androgens⁴⁰, and agents that increase CAMP including GLP-1³⁷. In addition, recently MIR-204, a highly β-cells enriched miRNA, has been shown to directly target the 3′ UTR of GLP1R, and downregulate its expression in INS-1 cells and primary mouse and human islets. Importantly, in vivo deletion of MIR-204 enhanced responsiveness to GLP1R agonists, resulting in improved glucose tolerance, insulin secretion, and protection against diabetes⁴¹. These findings demonstrate that GLP1R modifiers can impact key physiological processes in the maintenance of glucose homeostasis in vivo. Identifying molecular regulators of GLP1R expression may yield important new insight into factors underlying β-cell dysfunction and variation in response to GLP1R agonists and may inform the development of novel therapeutics to elevate GLP1R agonist efficacy.

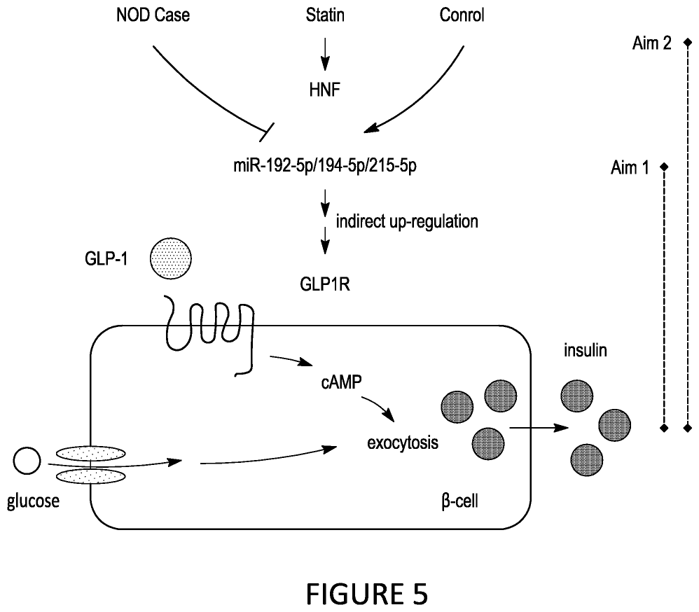

Using our unique resource of iPSCs from statin users who developed NOD (NOD cases) versus those who maintained normoglycemia (controls), we have identified MIR-192 as a novel indirect regulator of GLP1R that impact insulin secretion. FIG. 5 shows our working model where normally statin treatment up-regulates MIR-192 and therefore GLP1R, which enhances GSIS and promotes normal glycemic control. However, in some patients, statin treatment reduces MIR-192 and GLP1R, impaired insulin secretion, and greater risk for NOD.

### Example 2

### Investigation of miR-192 in the Regulation of GLP-1 Mediated Glucose Stimulated Insulin Secretion-PART A

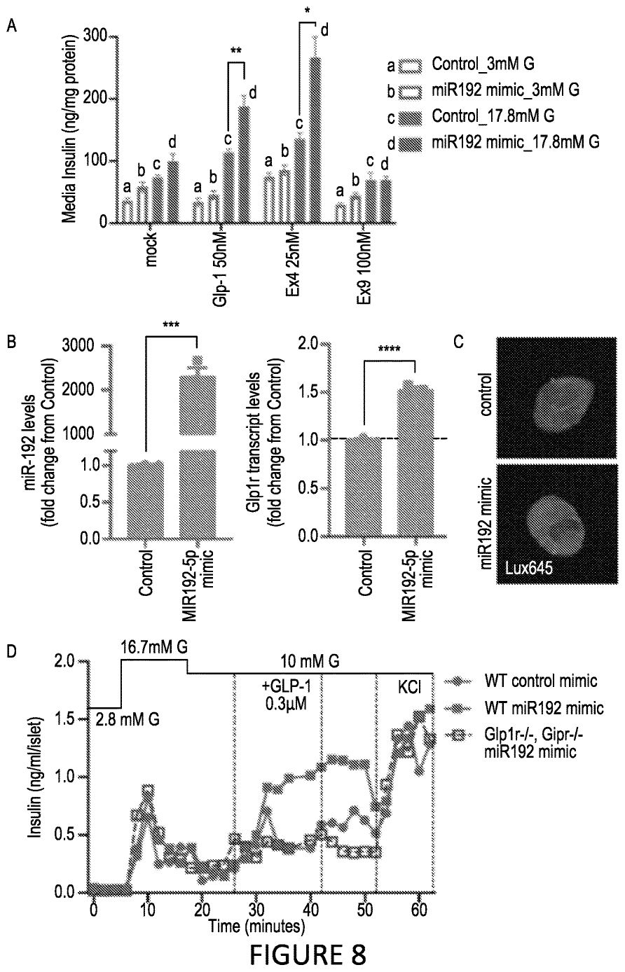

a-miR-192 increases GLP-1 stimulated insulin secretion Based on the increase in GLP-1R levels, we hypothesized that miR-192 may enhance the effects of GLP-1 on GSIS. In INS-1 miR-192 mimic treated cells, media insulin levels were elevated after stimulation with 17.8 mM glucose and 50 nM GLP-1 or 25 nM exendin-4 (a GLP-1R agonist) (FIG. 8A). In contrast, there was no effect on insulin levels with non-stimulatory glucose concentrations (3 mM) or after the addition of a GLP-1R antagonist (exendin9-39), demonstrating that miR-192 impacts GLP-1 potentiated GSIS through the GLP-1R.

We next sought to confirm miR-192 effects on GLP-1 potentiated GSIS in primary mouse islets. A collaborating laboratory has established a robust protocol to isolate and transfect primary murine islets for perifusion studies. Briefly, after a 16 hour fast, the pancreas was perfused with a collagenase solution injected into the common bile duct. Isolated islets were plated and repeatedly handpicked using dithizone staining as described to isolate 300 islets/mouse with a purity of 98-100%. Islets were then separated into single cell suspensions by 0.25% trypsin/EDTA, transfected with 80 nM of miR-192 mimic or control, and reaggregated by AggreWellTM400. 72 hr after transfection, we confirmed that both miR-192 and Glp1r transcript levels were increased (FIG. 8B), with a very similar magnitude of effect on Glp1r as seen in the INS-1 cells. We also observed increased GLP-1R protein levels by staining with LUXendin645, a highly specific fluorescent GLP-1R antagonistic peptide label obtained from other sources, FIG. 8C. Next, we measured GSIS by perifusion. We first confirmed that islets remained functional after reaggregation as evidenced by the distinct first and second phase of insulin secretion with high glucose (16.7 mM), and the transient and discreet increase observed upon the addition of 0.3 nM GLP-1 (FIG. 8D). In contrast, we identified a robust and sustained ~2-fold increase in GLP-1 mediated GSIS in the miR-192 mimic treated islets from wildtype animals, but not in islets from Glp1r/Gipr double knockout mice (a model we previously described^{42,43}). These findings demonstrate that miR-192 elevates GLP-1 potentiate GSIS through up regulation of GLP-1R.

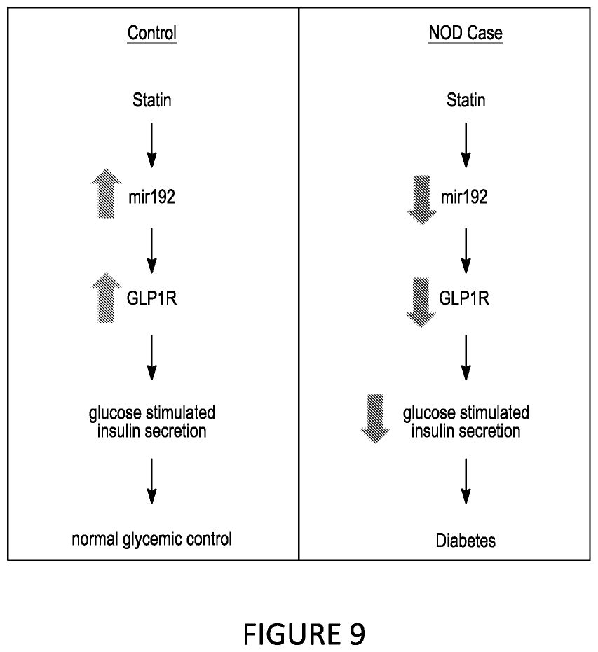

Notably, the transient increase in insulin secretion observed under control conditions when GLP-1 is abundant (perifusion studies lack the DDP-IV enzyme that normally degrades GLP-1) demonstrates that GLP-1R levels may be rate-liming. We hypothesize that the miR-192 mimic elevates GLP-1R levels, matching the higher availability of agonist, and enabling in a significantly longer duration of insulin secretion (2 vs. 18 minutes in miR-192 mimic treated cells, FIG. 8D. Extrapolating this observation to the clinical use of GLP-1R agonists, which have. half-lives on the order of hours⁴⁴, our findings demonstrate that upregulation of GLP-1R levels dramatically improve the efficacy of GLP-1R agonists. Lastly, these findings are consistent with our original observation that statin treatment has opposite effect on MIR-194-2HG and miR-192 levels between NOD cases vs. controls (FIG. 9). The increased miR-192 upon statin treatment in the controls would be expected to increase GLP-1R levels and enable GLP-1 potentiated GSIS and the maintenance of normoglycemia. In contrast, the reduction of miR-192 in the NOD cases would be expected to reduce GLP-1R levels and impair GLP-1 mediated GSIS, leading to the development of diabetes. To our knowledge, this observation may be the first identification of a molecular factor that contributes to inter-individual differences in the magnitude of statin induced dysglycemia.

### b-miR-192 Rescues Statin Induced Reductions in GLP-1 Mediated GSIS

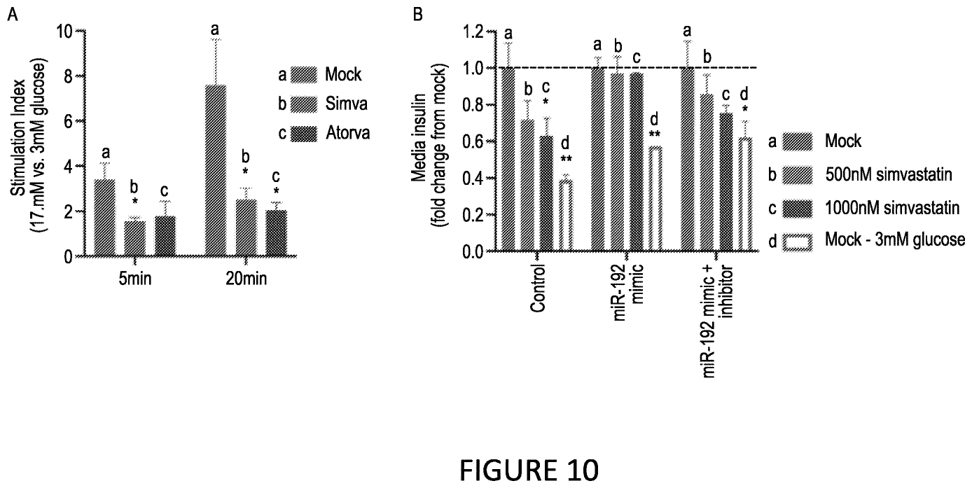

Consistent with multiple literature reports that statin treatment impairs insulin secretion^{63,65,66}, we found a reduction in the simulation index (FIG. 10A) as well as in the media insulin levels from INS-1 cells incubated with 17.8 mM glucose, 50 nM GLP-1 and statin (FIG. 10B). Importantly, transfection with the miR-192 mimic completely rescued the statin-induced reduction in media insulin levels, an effect that re-emerged in cells treated with the miR-192 mimic+inhibitor.

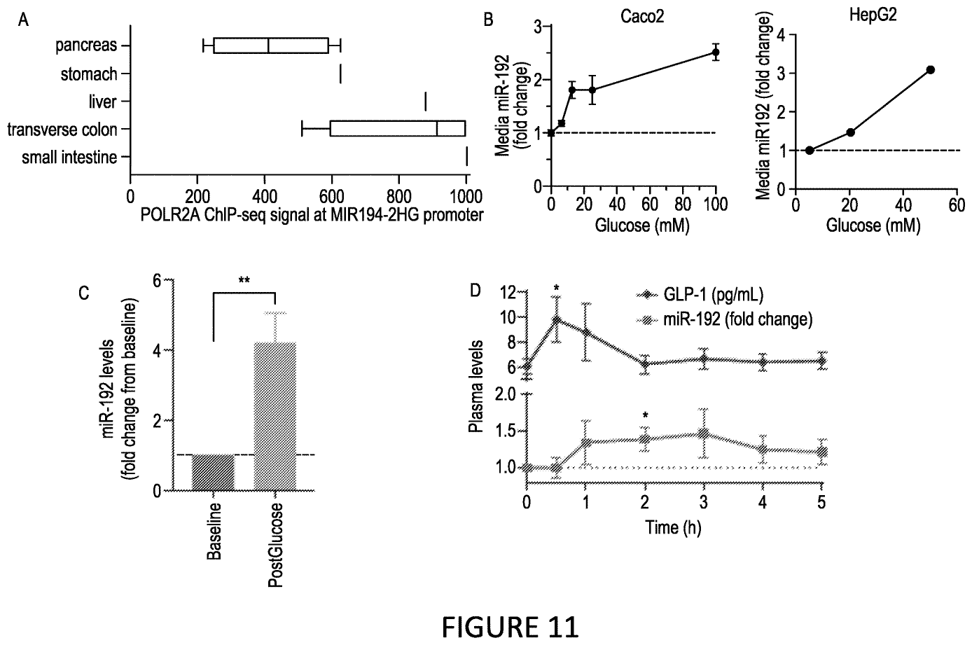

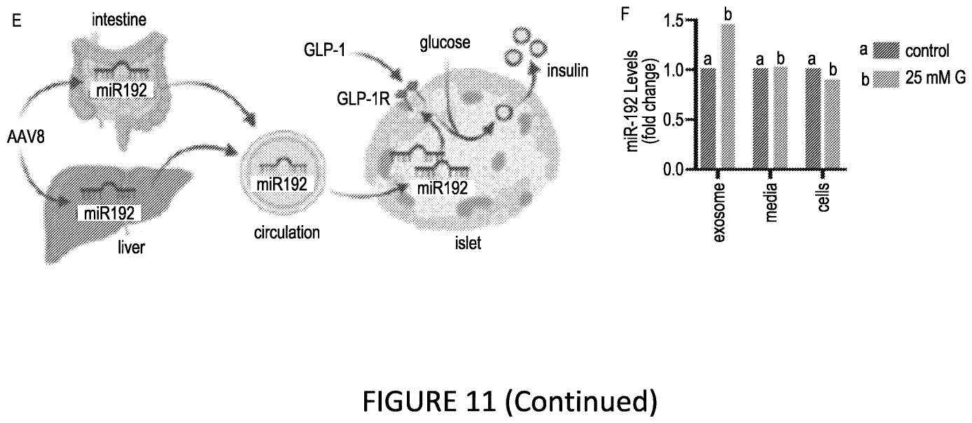

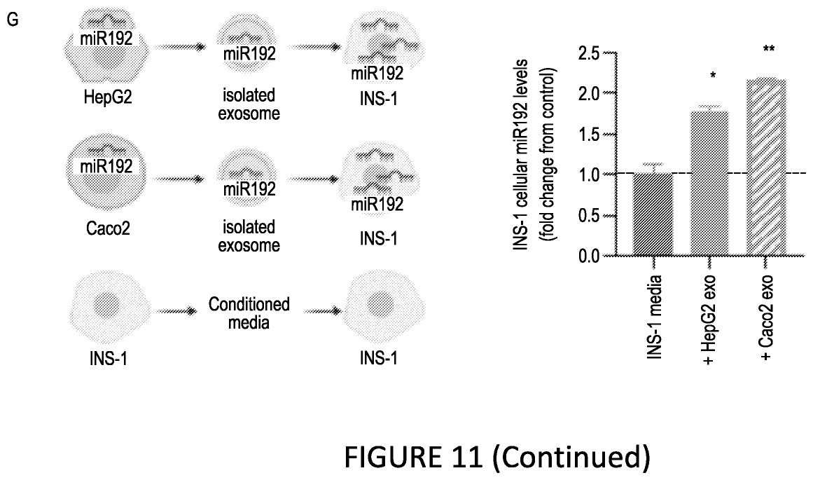

Effect of miR-192 in vivo. Based on bulk and single cell RNAseq in normal human and mouse tissues, miR-192 (precursor or mature) is expressed at a relatively low level in the pancreas^{67,60}, and is often described as specific to gastrointestinal tissues^{69,70}. To evaluate this further, we looked for evidence of RNA polymerase II binding (POLR2A) in the MIR-194-2HG promoter within in publicly available ENCODE data. Of the 81 various tissues represented, only 10 unique cell types had a detectable signal of POLR2A binding. Of these we found maximal signal in the small intestine with slightly lower levels in the colon and liver, and some signal in the pancreas (FIG. 11A), consistent with prior studies showing identification of miR-192 from intestine and liver-derived exosomes^{28,69,70}. We verified presence of miR-192 in Exoquick isolated exosomes from the media of Caco-2 (colorectal cell line) and HepG2 (hepatoma cell line), and found that media levels of miR-192 increased in a dose-dependent manner in response to glucose (FIG. 11B). In addition, we found that circulating miR-192 increased in portal vs. systemic blood within 15 minutes after an oral gavage in mice (FIG. 11C) as well as post-prandially in overweight women after a standardized meal⁷³ (FIG. 11D), consistent with miR-192 being secreted into the circulation from the intestine and/or liver. As serum levels of miR-192 are elevated in patients with T1D, T2D and NAFLD²⁰⁻²⁸ (where exosomal hepatic miR-192 secretion is increased²⁸) Based on our data showing increased β-cell function with miR-192 administration, miR-192 may be secreted from the liver and/or intestine as a means of metabolic cross-talk to elevate β-cell miR-192 and augment GLP-1 mediated GSIS (FIG. 11E). To query this model, we incubated INS-1 cells with exosomes containing miR-192 isolated from 25 mM glucose stimulated HepG2 and Caco2 cells and found higher intracellular levels of INS-1 miR-192 after incubation compared to control treated cells (FIGS. 11F, 11G).

c-miR-192 improves glucose sensitivity in murine models of T2D and statin-induced dysglycemia. The generation of a human T2D model is known in the literature⁸⁰. C57BL/6J male and female mice fed a 60% fat diet for 3 weeks and are treated with three once daily i.p. injections of 30 mg/kg streptozotocin. After 4 weeks, mice display β-cell dysfunction with reduced first and second phase insulin response to glucose and increased fed glucose levels. Like humans, the glycemic response to statin treatment in mice varies across different genetic backgrounds. A collaborator has generated a mouse model of statin-induced dysglycemia in which C57BL/6J animals are fed a simvastatin supplemented diet (calculated to produce an 80 mg/day human dose equivalent).

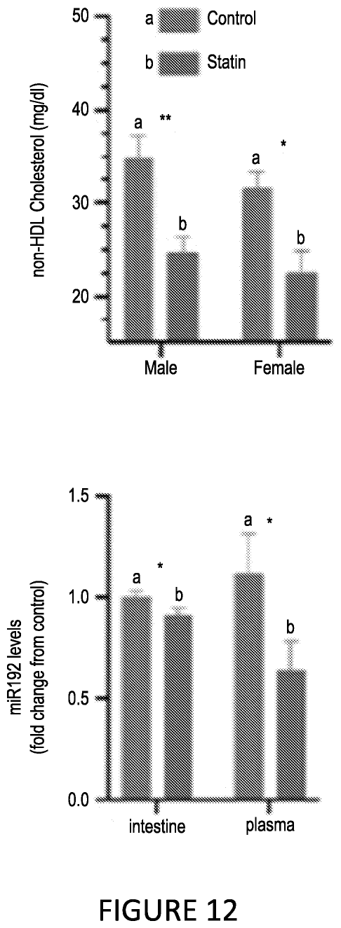

In our experiments, 12 weeks of statin feeding led to impaired glucose tolerance in both female and male animals and increased plasma fasting glucose levels in females. After 4 weeks we observed a reduction in non-HDL cholesterol and reduced miR-192 levels in the intestine and plasma with statin (FIG. 12), consistent with observations in NOD iPSCs.

### Example 3

### Investigation of miR-192 in the Regulation of GLP-1 Mediated Glucose Stimulated Insulin Secretion-PART B

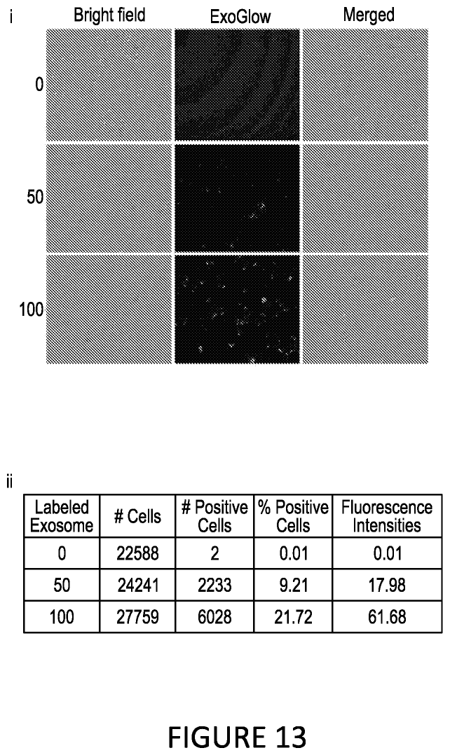

INS-1 uptake of HepG2 derived exosomes. Here it is demonstrated that miR-192 can be delivered to b cells from hepatic and/or intestinally derived exosomes as a means of augmenting GLP-1 mediated GSIS. In support of this, we have shown that exosomes isolated from conditioned media of 25 mM glucose treated HepG2 had higher levels of miR-192 then those from control HepG2, and that INS-1 cells incubated with exosomes from glucose treated HepG2 or Caco2 cells had higher intracellular miR-192 levels compared to control cells. In further experiments, we labeled exosomes isolated from HepG2 conditioned media with ExoGlow (System Biosciences), and incubated INS-1 with varying concentrations of the fluorescently labeled isolated exosomes. As shown in FIG. 13, we observed dose dependent uptake of labeled HepG2 exosomes by INS-1 cells. All studies were performed in cell (both HepG2 and INS-1 cells) incubated with exosome free FBS. These studies demonstrate that levels of miR-192 in the pancreas can be modulated through methods that do not directly target pancreatic tissue.

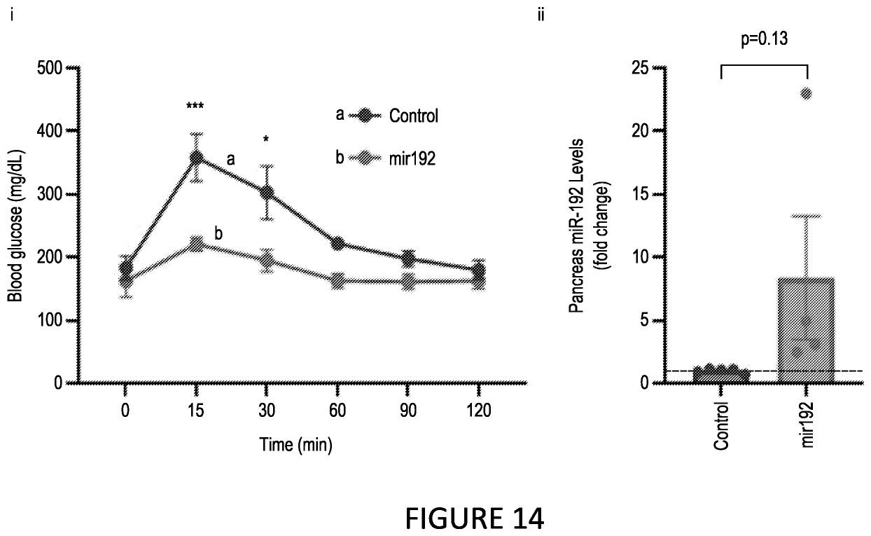

miR-192 AAV injected mice have higher pancreatic miR-192 levels and improved glucose tolerance. We evaluated the effect of miR-192 in vivo. These results demonstrate that increased levels of pancreatic miR-192 lead to improved glycemic control. Wildtype C57BL6/J male mice were fed a GAN diet (40 kcal % fat, 20 kcal % fructose and 2% cholesterol) at 7 weeks of age for 4 weeks before either AAV8-EF1a-miR-192 or control AAV vectors were delivered via tail vein injection. Animals remained on GAN diet for 4 weeks, during which there was no change in body weight gain between the miR-192 vs. control treated animals (data not shown). Four weeks after AAV injection, a glucose tolerance test (GTT) was performed in which each fasted animal was injected with 1.5 g/kg glucose via intraperitoneal injection and plasma glucose was quantified over 2 hours. Compared to animals treated with the control AAV (n=5), mice injected with miR-192 AAV (n=4) demonstrated dramatically improved glucose tolerance (FIG. 14.i), with significantly lower levels of plasma glucose at both the 15- and 30-minute time points. Importantly, all mice injected with miR-192 AAV had higher levels of pancreatic miR-192 than control mice (mean-8.4-fold, p=0.13), FIG. 14.ii). This difference is statistically significant if the mouse with the highest pancreatic miR-192 levels was removed (mean=3.5-fold, p=0.004), and remains significantly different when miR-192 levels were normalized to miR-16 levels (mean=3.3-fold, p=0.007). Together these findings demonstrate the feasibility of using indirect pancreatic miR-192 overexpression with systemically administered AAV8 vectors as well that increasing pancreatic miR-192 will improve glycemic control.

### REFERENCES A—CORRESPONDING TO CITATIONS IN EXAMPLE 1

1. Grundy, S. M. et al. 2018 AHA/ACC/AACVPR/AAPA/ABC/ACPM/ADA/AGS/APhA/ASPC/NLA/PCNA Guideline on the Management of Blood Cholesterol: A Report of the American College of Cardiology/American Heart Association Task Force on Clinical Practice Guidelines. Circulation 139, E1082-E1143 (2019).
2. D I, S. et al. HMG-coenzyme A reductase inhibition, type 2 diabetes, and bodyweight: evidence from genetic analysis and randomised trials. Lancet (London, England) 385, 351-361 (2015).
3. M O, G., X, L., R M, K., J I, R. & Y D, C. Relationship of sex to diabetes risk in statin trials. Diabetes Care 36, (2013).
4. P M, R., A, P., J G, M., P, L. & R J, G. Cardiovascular benefits and diabetes risks of statin therapy in primary prevention: an analysis from the JUPITER trial. Lancet (London, England) 380, 565-571 (2012).
5. P L, B. & D J, D. Minireview: Glucagon-like peptides regulate cell proliferation and apoptosis in the pancreas, gut, and central nervous system. Endocrinology 145, 2653-2659 (2004).
6. L L, B. & D J, D. Biology of incretins: GLP-1 and GIP. Gastroenterology 132, 2131-2157 (2007).
7. Y, L. & P D, R. Glucagon-Like Peptide-1 Receptor Agonists and Cardiovascular Risk Reduction in Type 2 Diabetes Mellitus: Is It a Class Effect? Curr. Cardiol. Rep. 20, (2018).
8. D, H. Glucagon-Like Peptide 1 Receptor Agonists for Type 2 Diabetes. Diabetes Spectr. 30, 202-210 (2017).
9. V, S. et al. Simvastatin Rapidly and Reversibly Inhibits Insulin Secretion in Intact Single-Islet Cultures. Diabetes Ther. 7, 679-693 (2016).
10. F, U. et al. Atorvastatin but Not Pravastatin Impairs Mitochondrial Function in Human Pancreatic Islets and Rat B-Cells. Direct Effect of Oxidative Stress. Sci. Rep. 7, (2017).
11. N, Y. et al. Simvastatin Impairs Insulin Secretion by Multiple Mechanisms in MIN6 Cells. PLOS One 10, (2015).
12. A, J. et al. Circulating microRNAs-192 and −194 are associated with the presence and incidence of diabetes mellitus. Sci. Rep. 8, (2018).
13. Pan, W. et al. MiR-192 is upregulated in T1D M, regulates pancreatic β-cell development and inhibits insulin secretion through suppressing GLP-1 expression. Exp. Ther. Med. 16, 2717-2724 (2018).
14. Y L, K. et al. Evaluation of commonly used ectoderm markers in iPSC trilineage differentiation. Stem Cell Res. 37, (2019).
15. Okita, K. et al. An efficient nonviral method to generate integration-free human-induced pluripotent stem cells from cord blood and peripheral blood cells. Stem Cells 31, 458-466 (2013).
16. Avior, Y., Sagi, I. & Benvenisty, N. Pluripotent stem cells in disease modelling and drug discovery. Nat Rev Mol Cell Biol 17, 170-182 (2016).
17. Sterneckert, J. L., Reinhardt, P. & Scholer, H. R. Investigating human disease using stem cell models. Nat Rev Genet 15, 625-639 (2014).
18. Medina, M. W. et al. RHOA Is a Modulator of the Cholesterol-Lowering Effects of Statin. PLOS Genet. 8, (2012).
19. B, D., M, W., A, C., H, L. & J, L. Suppression of Idol expression is an additional mechanism underlying statin-induced up-regulation of hepatic LDL receptor expression. Int. J. Mol. Med. 27, 103-110 (2011).
20. J L, G. & M S, B. Regulation of the mevalonate pathway. Nature 343, 425-430 (1990).
21. S, E., A, M., R, T., C, P. & T J, K. Profiling of circulating microRNAs in children with recent onset of type 1 diabetes. JCI insight 2, (2017).
22. M, P. et al. Circulating miR-192 and miR-193b are markers of prediabetes and are modulated by an exercise intervention. J. Clin. Endocrinol. Metab. 100, E407-E415 (2015).
23. Z, Y. et al. Serum miR-23a, a potential biomarker for diagnosis of pre-diabetes and type 2 diabetes. Acta Diabetol. 51, 823-831 (2014).
24. Jia, Y. et al. Exendin-4 ameliorates high glucose-induced fibrosis by inhibiting the secretion of miR-192 from injured renal tubular epithelial cells. Exp. Mol. Med. 50, (2018).
25. N, L. et al. Distribution of miRNA expression across human tissues. Nucleic Acids Res. 44, 3865-3877 (2016).
26. M, van de B. et al. The miRNA profile of human pancreatic islets and beta-cells and relationship to type 2 diabetes pathogenesis. PLOS One 8, (2013).
27. J, O., H, H., Y, Z. & C, P. Overview of MicroRNA Biogenesis, Mechanisms of Actions, and Circulation. Front. Endocrinol. (Lausanne). 9, (2018).
28. X L, L. et al. miR-192-5p regulates lipid synthesis in non-alcoholic fatty liver disease through SCD-1. World J. Gastroenterol. 23, 8140-8151 (2017).
29. Cosentino, F. et al. 2019 ESC Guidelines on diabetes, pre-diabetes, and cardiovascular diseases developed in collaboration with the EASD The Task Force for diabetes, pre-diabetes, and cardiovascular diseases of the European Society of Cardiology (ESC) and the European Association for the Study of Diabetes (EASD). Eur. Heart J. 41, 255-323 (2020).
30. M, K., M, T., H, I. & Y, S. Glucose-stimulated insulin secretion: A newer perspective. J. Diabetes Investig. 4, 511-516 (2013).
31 M A, N., E, B., C, O., R, E. & W, C. Additive insulinotropic effects of exogenous synthetic human gastric inhibitory polypeptide and glucagon-like peptide-1-(7-36) amide infused at near-physiological insulinotropic hormone and glucose concentrations. J. Clin. Endocrinol. Metab. 76, 912-917 (1993).
32 M A, N. et al. Preserved incretin activity of glucagon-like peptide 1 [7-36 amide] but not of synthetic human gastric inhibitory polypeptide in patients with type-2 diabetes mellitus. J. Clin. Invest. 91, 301-307 (1993).
33. M P, G. & R E, P. GLP-1 Analogs and DPP-4 Inhibitors in Type 2 Diabetes Therapy: Review of Head-to-Head Clinical Trials. Front. Endocrinol. (Lausanne). 11, (2020).
34. G B, L. GLP1R agonists: primary cardiovascular prevention and oral administration. Nat. Rev. Cardiol. 16, 453 (2019).
35. T R, E., A, A., C, L. & U H, P. Prevalence of cardiovascular disease in type 2 diabetes: a systematic literature review of scientific evidence from across the world in 2007-2017. Cardiovasc. Diabetol. 17, (2018).
36 P, N., O G, C. & G G, H. Regulation of glucose homeostasis by GLP-1. Prog. Mol. Biol. Transl. Sci. 121, 23-65 (2014).
37. H C, F., J, J., D, P., J, S. & B, G. Ligand-induced regulation of glucagon-like peptide-I receptor function and expression in insulin-secreting beta cells. Pancreas 13, 273-282 (1996).
38. G M, W., F C, L., C H, M. & E A, A. Regulation of GIP and GLP1 receptor cell surface expression by N-glycosylation and receptor heteromerization. PLOS One 7, (2012).
39. N, A. & E, N. Regulation of glucagon and glucagon-like peptide-1 receptor messenger ribonucleic acid expression in cultured rat pancreatic islets by glucose, cyclic adenosine 3′,5′-monophosphate, and glucocorticoids. Endocrinology 136, 1572-1578 (1995).
40. L, Z. et al. Glucagon-like peptide-1 receptor expression and its functions are regulated by androgen. Biomed. Pharmacother. 120, (2019).
41. S, J. et al. miR-204 Controls Glucagon-Like Peptide 1 Receptor Expression and Agonist Function. Diabetes 67, 256-264 (2018).
42. Marx et al., GLP-1 Receptor Agonists for the Reduction of Atherosclerotic Cardiovascular Risk in Patients With Type 2 Diabetes. Circulation 146 (24): 1882-1894 (2022).

### REFERENCES B—CORRESPONDING TO CITATION IN EXAMPLE 2

1. Frias, J. P. et al. Tirzepatide versus Semaglutide Once Weekly in Patients with Type 2 Diabetes. The New England journal of medicine 385, 503-515, doi:10.1056/NEJMoa2107519 (2021).
2. 2 Frias, J. P. et al. Efficacy and safety of LY3298176, a novel dual GIP and GLP-1 receptor agonist, in patients with type 2 diabetes: a randomised, placebo-controlled and active comparator-controlled phase 2 trial. Lancet 392, 2180-2193, doi:10.1016/S0140-6736 (18) 32260-8 (2018).
3. 3 Giugliano, D. et al. GLP-1 receptor agonists and cardiorenal outcomes in type 2 diabetes: an updated meta-analysis of eight CVOTs. Cardiovascular diabetology 20, 189, doi:10.1186/s12933-021-01366-8 (2021). PMC8442438
4. 4 Lavalle-Cobo, A., Masson, W., Lobo, M., Masson, G. & Molinero, G. Glucagon-like Peptide-1 Receptor Agonists and Cardioprotective Benefit in Patients with Type 2 Diabetes Without Baseline Metformin: A Systematic Review and Update Meta-analysis. High Blood Press Cardiovasc Prev 28, 605-612, doi:10.1007/s40292-021-00479-1 (2021).
5. 5 Al Rifai, M. et al. Correlates of Glucagon-Like Peptide-1 Receptor Agonist Use Among Patients With Atherosclerotic Cardiovascular Disease and Type 2 Diabetes Mellitus (from the Department of Veterans Affairs). Am J Cardiol, doi:10.1016/j.amjcard.2022.02.013 (2022).
6. 6 Drucker, D. J. Mechanisms of Action and Therapeutic Application of Glucagon-like Peptide-1. Cell Metab 27, 740-756, doi:10.1016/j.cmet.2018.03.001 (2018).
7. 7 Nauck, M. A., Quast, D. R., Wefers, J. & Meier, J. J. GLP-1 receptor agonists in the treatment of type 2 diabetes-state-of-the-art. Mol Metab 46, 101102, doi:10.1016/j.molmet.2020.101102 (2021). PMC8085572
8. 8 Smith, E. P. et al. The role of beta cell glucagon-like peptide-1 signaling in glucose regulation and response to diabetes drugs. Cell Metab 19, 1050-1057, doi:10.1016/j.cmet.2014.04.005 (2014). PMC4109713
9. 9 Capozzi, M. E. et al. beta Cell tone is defined by proglucagon peptides through cAMP signaling. JCI Insight 4, doi:10.1172/jci.insight. 126742 (2019). PMC6483521
10. 10 Capozzi, M. E. et al. Glucagon lowers glycemia when beta-cells are active. JCI Insight 5, doi:10.1172/jci.insight. 129954 (2019). PMC6777806
11. 11 E I, K. et al. GIP mediates the incretin effect and glucose tolerance by dual actions on alpha cells and beta cells. Sci Adv 7, doi:10.1126/sciadv.abf1948 (2021). PMC7954443
12. 12 Svendsen, B. et al. Insulin Secretion Depends on Intra-islet Glucagon Signaling. Cell Rep 25, 1127-1134 e1122, doi:10.1016/j.celrep.2018.10.018 (2018).
13. 13 Zhu, L. et al. Intra-islet glucagon signaling is critical for maintaining glucose homeostasis. JCI Insight 5, doi:10.1172/jci.insight.127994 (2019). PMC6542600
14. 14 Lamont, B. J. et al. Pancreatic GLP-1 receptor activation is sufficient for incretin control of glucose metabolism in mice. The Journal of clinical investigation 122, 388-402, doi:10.1172/JCI42497 (2012). PMC3248276
15. 15 Shu, L. et al. Decreased TCF7L2 protein levels in type 2 diabetes mellitus correlate with downregulation of GIP- and GLP-1 receptors and impaired beta-cell function. Hum Mol Genet 18, 2388-2399, doi:10.1093/hmg/ddp178 (2009). PMC2722186
16. 16 Vujkovic, M. et al. Discovery of 318 new risk loci for type 2 diabetes and related vascular outcomes among 1.4 million participants in a multi-ancestry meta-analysis. Nat Genet 52, 680-691, doi:10.1038/s41588-020-0637-y (2020). PMC7343592
17. 17 Consortium, G. T. The GTEx Consortium atlas of genetic regulatory effects across human tissues. Science 369, 1318-1330, doi:10.1126/science.aaz1776 (2020). PMC7737656
18. 18 Nadkarni, P., Chepurny, O. G. & Holz, G. G. Regulation of glucose homeostasis by GLP-1. Prog Mol Biol Transl Sci 121, 23-65, doi:10.1016/B978-0-12-800101-1.00002-8 (2014). PMC4159612
19. 19 Jo, S. et al. miR-204 Controls Glucagon-Like Peptide 1 Receptor Expression and Agonist Function. Diabetes 67, 256-264, doi:10.2337/db17-0506 (2018). PMC5780066
20. 20 Ren, F. J., Yao, Y., Cai, X. Y. & Fang, G. Y. Emerging Role of MIR-192-5p in Human Diseases. Front Pharmacol 12, 614068, doi:10.3389/fphar.2021.614068 (2021). PMC7940509
21. 21 Jaeger, A. et al. Circulating microRNAs-192 and -194 are associated with the presence and incidence of diabetes mellitus. Sci Rep 8, 14274, doi:10.1038/s41598-018-32274-9 (2018). PMC6155281
22. 22 Lischka, J. et al. Circulating microRNAs 34a, 122, and 192 are linked to obesity-associated inflammation and metabolic disease in pediatric patients. Int J Obes (Lond), doi:10.1038/s41366-021-00842-1 (2021).
23. 23 Pan, W. et al. miR-192 is upregulated in T1D M, regulates pancreatic beta-cell development and inhibits insulin secretion through suppressing GLP-1 expression. Exp Ther Med 16, 2717-2724, doi:10.3892/etm.2018.6453 (2018). PMC6122452
24. 24 Parrizas, M. et al. Circulating miR-192 and miR-193b are markers of prediabetes and are modulated by an exercise intervention. The Journal of clinical endocrinology and metabolism 100, E407-415, doi:10.1210/jc.2014-2574 (2015).
25. 25 Erener, S., Marwaha, A., Tan, R., Panagiotopoulos, C. & Kieffer, T. J. Profiling of circulating microRNAs in children with recent onset of type 1 diabetes. JCI Insight 2, e89656, doi:10.1172/jci.insight.89656 (2017). PMC5313074 exists.
26. 26 Yang, Z. et al. Serum miR-23a, a potential biomarker for diagnosis of pre-diabetes and type 2 diabetes. Acta diabetologica 51, 823-831, doi:10.1007/s00592-014-0617-8 (2014).
27. 27 Liu, X. L. et al. miR-192-5p regulates lipid synthesis in non-alcoholic fatty liver disease through SCD-1. World J Gastroenterol 23, 8140-8151, doi:10.3748/wjg.v23.i46.8140 (2017). PMC5739921
28. 28 Liu, X. L. et al. Lipotoxic Hepatocyte-Derived Exosomal MicroRNA 192-5p Activates Macrophages Through Rictor/Akt/Forkhead Box Transcription Factor 01 Signaling in Nonalcoholic Fatty Liver Disease. Hepatology 72, 454-469, doi:10.1002/hep.31050 (2020).
29. 29 Castano, C., Kalko, S., Novials, A. & Parrizas, M. Obesity-associated exosomal miRNAs modulate glucose and lipid metabolism in mice. Proc Natl Acad Sci USA 115, 12158-12163, doi:10.1073/pnas. 1808855115 (2018). PMC6275521
30. 30 Flowers, E., Allen, I. E., Kanaya, A. M. & Aouizerat, B. E. Circulating microRNAs are associated with variability in fasting blood glucose over 12-months and target pathways related to type 2 diabetes: A pilot study. Diab Vasc Dis Res 18, 14791641211055837, doi:10.1177/14791641211055837 (2021). PMC8761879
31. 31 Ruan, Y. et al. Circulating LncRNAs Analysis in Patients with Type 2 Diabetes Reveals Novel Genes Influencing Glucose Metabolism and Islet beta-Cell Function. Cell Physiol Biochem 46, 335-350, doi:10.1159/000488434 (2018).
32. 32 Flammang, I., Reese, M., Yang, Z., Eble, J. A. & Dhayat, S. A. Tumor-Suppressive miR-192-5p Has Prognostic Value in Pancreatic Ductal Adenocarcinoma. Cancers (Basel) 12, doi:10.3390/cancers12061693 (2020). PMC7352756
33. 33 Short, K. R. et al. in Diabetes Vol. 70 (Supplement 1) (2021).
34. 34 Kim, J., Lee, H. S. & Lee, K. Y. Effect of statins on fasting glucose in non-diabetic individuals:
   nationwide population-based health examination in Korea. Cardiovascular diabetology 17, 155,
   doi:10.1186/s12933-018-0799-4 (2018). PMC6280428
35. 35 Kuang, Y. L. et al. Evaluation of commonly used ectoderm markers in iPSC trilineage differentiation.
   Stem Cell Res 37, 101434, doi:10.1016/j.scr.2019.101434 (2019). PMC6570500
36. 36 Medina, M. W. et al. RHOA is a modulator of the cholesterol-lowering effects of statin. PLOS Genet 8, e1003058, doi:10.1371/journal.pgen.1003058 (2012). PMC3499361
37. 37 Dong, B., Wu, M., Cao, A., Li, H. & Liu, J. Suppression of Idol expression is an additional mechanism underlying statin-induced up-regulation of hepatic LDL receptor expression. International journal of molecular medicine 27, 103-110, doi:10.3892/ijmm.2010.559 (2011).
38. 38 Goldstein, J. L. & Brown, M. S. Regulation of the mevalonate pathway. Nature 343, 425-430 (1990).
39. 39 O'Brien, J., Hayder, H., Zayed, Y. & Peng, C. Overview of MicroRNA Biogenesis, Mechanisms of Actions, and Circulation. Front Endocrinol (Lausanne) 9, 402, doi:10.3389/fendo.2018.00402 (2018). PMC6085463
40. 40 Mysore, R. et al. MicroRNA-192* impairs adipocyte triglyceride storage. Biochim Biophys Acta 1861, 342-351, doi:10.1016/j.bbalip.2015.12.019 (2016).
41. 41 Saliba, Y., Bakhos, J. J., Itani, T. & Fares, N. An optimized protocol for purification of functional islets of Langerhans. Lab Invest 97, 70-83, doi:10.1038/labinvest.2016.123 (2017).
42. 42 Hansotia, T. et al. Double incretin receptor knockout (DIRKO) mice reveal an essential role for the enteroinsular axis in transducing the glucoregulatory actions of DPP-IV inhibitors. Diabetes 53, 1326-1335, doi:10.2337/diabetes.53.5.1326 (2004).
43. 43 Ayala, J. E. et al. Insulin action in the double incretin receptor knockout mouse. Diabetes 57, 288-297, doi:10.2337/db07-0704 (2008).
44. 44 Garber, A. J. Long-acting glucagon-like peptide 1 receptor agonists: a review of their efficacy and tolerability. Diabetes Care 34 Suppl 2, S279-284, doi:10.2337/dc11-s231 (2011). PMC3632193
45. 45 Dobin, A. et al. STAR: ultrafast universal RNA-seq aligner. Bioinformatics 29, 15-21, doi:10.1093/bioinformatics/bts635 (2013). PMC3530905
46. 46 Frankish, A. et al. GENCODE reference annotation for the human and mouse genomes. Nucleic Acids Res 47, D766-D773, doi:10.1093/nar/gky955 (2019). PMC6323946
47. 47 Liao, Y., Smyth, G. K. & Shi, W. featureCounts: an efficient general purpose program for assigning sequence reads to genomic features. Bioinformatics 30, 923-930, doi:10.1093/bioinformatics/btt656 (2014).
48. 48 Love, M. I., Huber, W. & Anders, S. Moderated estimation of fold change and dispersion for RNA-seq data with DESeq2. Genome Biol 15, 550, doi:10.1186/s13059-014-0550-8 (2014). PMC4302049
49. 49 Agarwal, V., Bell, G. W., Nam, J. W. & Bartel, D. P. Predicting effective microRNA target sites in mammalian mRNAs. Elife 4, doi:10.7554/eLife.05005 (2015). PMC4532895
50. 50 Chen, Y. & Wang, X. miRDB: an online database for prediction of functional microRNA targets. Nucleic Acids Res 48, D127-D131, doi:10.1093/nar/gkz757 (2020). PMC6943051
51. 51 Bandyopadhyay, S. & Mitra, R. TargetMiner: microRNA target prediction with systematic identification of tissue-specific negative examples. Bioinformatics 25, 2625-2631, doi:10.1093/bioinformatics/btp503 (2009).
52. 52 Paraskevopoulou, M. D. et al. DIANA-microT web server v5.0: service integration into miRNA functional analysis workflows. Nucleic Acids Res 41, W169-173, doi:10.1093/nar/gkt393 (2013). PMC3692048
53. 53 Krek, A. et al. Combinatorial microRNA target predictions. Nat Genet 37, 495-500, doi:10.1038/ng1536 (2005).
54. 54 Xiao, Y. et al. A Novel Interacting Protein SERP1 Regulates the N-Linked Glycosylation and Function of GLP-1 Receptor in the Liver. Journal of cellular biochemistry 118, 3616-3626, doi:10.1002/jcb.26207 (2017).
55. 55 Ast, J. et al. Super-resolution microscopy compatible fluorescent probes reveal endogenous glucagon-like peptide-1 receptor distribution and dynamics. Nature communications 11, 467, doi:10.1038/s41467-020-14309-w (2020). PMC6981144
56. 56 Qin, Y. et al. Phosphatidylinositol-(4,5)-Bisphosphate Regulates Plasma Cholesterol Through LDL (Low-Density Lipoprotein) Receptor Lysosomal Degradation. Arterioscler Thromb Vasc Biol 40, 1311-1324, doi:10.1161/ATVBAHA. 120.314033 (2020). PMC7197750
57. 57 Dai, F. F. et al. A Novel GLP1 Receptor Interacting Protein ATP6ap2 Regulates Insulin Secretion in Pancreatic Beta Cells. J Biol Chem 290, 25045-25061, doi:10.1074/jbc.M115.648592 (2015). PMC4599009
58. 58 Sharma, S. Isolation of a sequence-specific RNA binding protein, polypyrimidine tract binding protein, using RNA affinity chromatography. Methods in molecular biology 488, 1-8, doi:10.1007/978-1-60327-475-3_1 (2008).
59. 59 Kretz, M. et al. Control of somatic tissue differentiation by the long non-coding RNA TINCR. Nature 493, 231-235, doi:10.1038/nature11661 (2013). PMC3674581
60. 60 Oni-Orisan, A. et al. Characterization of Statin Low-Density Lipoprotein Cholesterol Dose-Response Using Electronic Health Records in a Large Population-Based Cohort. Circ Genom Precis Med 11, e002043, doi:10.1161/CIRCGEN.117.002043 (2018). PMC6214660
61. 61 Trube, G., Rorsman, P. & Ohno-Shosaku, T. Opposite effects of tolbutamide and diazoxide on the ATP-dependent K+ channel in mouse pancreatic beta-cells. Pflugers Arch 407, 493-499, doi:10.1007/BF00657506 (1986).
62. 62 Leippe, D., Sobol, M., Vidugiris, G., Cali, J. J. & Vidugiriene, J. Bioluminescent Assays for Glucose and Glutamine Metabolism: High-Throughput Screening for Changes in Extracellular and Intracellular Metabolites. SLAS Discov 22, 366-377, doi:10.1177/1087057116675612 (2017).
63. 63 Urbano, F. et al. Atorvastatin but Not Pravastatin Impairs Mitochondrial Function in Human Pancreatic Islets and Rat beta-Cells. Direct Effect of Oxidative Stress. Sci Rep 7, 11863, doi:10.1038/s41598-017-11070-x (2017). PMC5605712
64. 64 Hamanaka, R. B. & Chandel, N. S. Mitochondrial reactive oxygen species regulate cellular signaling and dictate biological outcomes. Trends Biochem Sci 35, 505-513, doi:10.1016/j.tibs.2010.04.002 (2010). PMC2933303
65. 65 Scattolini, V. et al. Simvastatin Rapidly and Reversibly Inhibits Insulin Secretion in Intact Single-Islet Cultures. Diabetes Ther 7, 679-693, doi:10.1007/s13300-016-0210-y (2016). PMC5118248
66. 66 Yaluri, N. et al. Simvastatin Impairs Insulin Secretion by Multiple Mechanisms in MIN6 Cells. PLOS One 10, e0142902, doi:10.1371/journal.pone.0142902 (2015). PMC4641640
67. 67 Consortium, G. T. Human genomics. The Genotype-Tissue Expression (GTEx) pilot analysis: multitissue gene regulation in humans. Science 348, 648-660, doi:10.1126/science. 1262110 (2015).
68. 68 Tabula Muris, C. et al. Single-cell transcriptomics of 20 mouse organs creates a Tabula Muris. Nature 562, 367-372, doi:10.1038/s41586-018-0590-4 (2018). PMC6642641
69. 69 Liang, Y., Ridzon, D., Wong, L. & Chen, C. Characterization of microRNA expression profiles in normal human tissues. BMC Genomics 8, 166, doi:10.1186/1471-2164-8-166 (2007). PMC1904203
70. 70 Gotanda, K. et al. Circulating intestine-derived exosomal miR-328 in plasma, a possible biomarker for estimating BCRP function in the human intestines. Sci Rep 6, 32299, doi:10.1038/srep32299 (2016). PMC5004159
71. 71 Gil-Zamorano, J. et al. Docosahexaenoic acid modulates the enterocyte Caco-2 cell expression of microRNAs involved in lipid metabolism. J Nutr 144, 575-585, doi:10.3945/jn.113.189050 (2014).
72. 72 Jia, Y. et al. Exendin-4 ameliorates high glucose-induced fibrosis by inhibiting the secretion of miR-192 from injured renal tubular epithelial cells. Exp Mol Med 50, 1-13, doi:10.1038/s12276-018-0084-3 (2018). PMC5938044
73. 73 Lesser, M. N. R., Mauldin, K., Sawrey-Kubicek, L., Gildengorin, V. & King, J. C. The Type of Dietary Fat in an Isocaloric Breakfast Meal Does Not Modify Postprandial Metabolism in Overweight/Obese Pregnant Women. Nutrients 11, doi:10.3390/nu11030490 (2019). PMC6471094
74. 74 Brown, R. A. M. et al. Evaluation of MicroRNA Delivery In Vivo. Methods in molecular biology 1699, 155-178, doi:10.1007/978-1-4939-7435-1_12 (2018).
75. 75 Polyak, S. et al. Identification of adeno-associated viral vectors suitable for intestinal gene delivery and modulation of experimental colitis. American journal of physiology. Gastrointestinal and liver physiology 302, G296-308, doi:10.1152/ajpgi.00562.2010 (2012).
76. 76 Ayala, J. E. et al. Glucagon-like peptide-1 receptor knockout mice are protected from high-fat diet-induced insulin resistance. Endocrinology 151, 4678-4687, doi:10.1210/en.2010-0289 (2010). PMC2946144
77. 77 Tong, J. & D'Alessio, D. Give the receptor a brake: slowing gastric emptying by GLP-1. Diabetes 63, 407-409, doi:10.2337/db13-1764 (2014).
78. 78 Varin, E. M. et al. Distinct Neural Sites of GLP-1R Expression Mediate Physiological versus Pharmacological Control of Incretin Action. Cell Rep 27, 3371-3384 e3373, doi:10.1016/j.celrep.2019.05.055 (2019).
79. 79 Gabery, S. et al. Semaglutide lowers body weight in rodents via distributed neural pathways. JCI Insight 5, doi:10.1172/jci.insight.133429 (2020). PMC7213778
80. 80 Parilla, J. H., Willard, J. R., Barrow, B. M. & Zraika, S. A Mouse Model of Beta-Cell Dysfunction as Seen in Human Type 2 Diabetes. Journal of diabetes research 2018, 6106051, doi:10.1155/2018/6106051 (2018). PMC5952555
81. 81 Elzinga, S. E. et al. Sex differences in insulin resistance, but not peripheral neuropathy, in a diet-induced prediabetes mouse model. Disease models & mechanisms 14, doi:10.1242/dmm.048909 (2021). PMC8077554
82. 82 Goodarzi, M. O., Li, X., Krauss, R. M., Rotter, J. I. & Chen, Y. D. Relationship of sex to diabetes risk in statin trials. Diabetes Care 36, e100-101, doi:10.2337/dc13-0490 (2013). 3687308
83. 83 Henquin, J. C., Nenquin, M., Stiernet, P. & Ahren, B. In vivo and in vitro glucose-induced biphasic insulin secretion in the mouse: pattern and role of cytoplasmic Ca2+ and amplification signals in beta-cells. Diabetes 55, 441-451, doi:10.2337/diabetes.55.02.06.db05-1051 (2006).

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

What is claimed is:

1. A method of achieving a selected therapeutic outcome in a subject by administration to the subject of a therapeutically effective amount of an miR-192 agent.

2. The method of claim 1, wherein the miR-192 agent comprises an miR-192 nucleic acid sequence, sequence variant thereof, or chemical mimic thereof.

3. The method of claim 2, wherein the miR-192 agent comprises at least 90% sequence identity to hsa-miR-192-5p (SEQ ID NO: 1).

4. The method of claim 3, wherein the nucleic acid is packaged in an extracellular vesicle.

5. The method of claim 1, wherein the miR-192 agent comprises a transformation vector comprising an miR-192 nucleic acid sequence.

6. The method of claim 5, wherein the transformation vector expresses a sequence comprising at least 90% sequence identity to hsa-miR-192-5p (SEQ ID NO: 1).

7. The method of any one of claims 1-6, wherein the miR-192 agent is administered systemically.

8. The method of any one of claims 1-7, wherein the selected therapeutic outcome is increasing GSIS.

9. The method of any one of claims 1-8, wherein the selected therapeutic outcome is increasing GLP1 activity.

10. The method of any one of claims 1-9, wherein the selected therapeutic outcome is increasing GLP1R abundance and/or activity.

11. The method of any one of claims 1-10, wherein the selected therapeutic outcome is weight loss.

12. The method of any one of claims 1-11, wherein the selected therapeutic outcome is treatment of Type II diabetes.

13. The method of any one of claims 1-11, wherein the selected therapeutic outcome is the prevention of new onset diabetes.

14. The method of claim 13, wherein the subject is a statin user.

15. The method of any one of claims 1-14, wherein the selected therapeutic outcome is treatment or prevention of a cardiovascular disease.

16. The method of claim 15, wherein the cardiovascular disease is atherosclerotic cardiovascular disease.

17. The method of any of claims 6-16, wherein the MiR-192 agent is co-administered with a GLP-1 agonist or DPP-IV inhibitor.

18. A method of assessing elevated risk of statin-induced NOD in a subject, comprising the steps:
a first sample is obtained from a subject that is not using statins;
miR-192 family member abundance in the sample is measured;
a second sample is obtained from the subject after they have been administered statins;
miR-192 family member abundance in the sample measured;
the first and second measurements of miR-192 family member abundance are compared; and
risk of statin-induced NOD is assessed, wherein a decrease in miR-192 family member abundance after statin use is indicative of elevated risk of statin-induced NOD.

19. A method of assessing elevated risk of developing statin-induced NOD in a subject, comprising the steps:
a cellular sample is obtained from a subject;
a cell culture derived from the sample is established;
miR-192 family member abundance in the cells is measured in the absence of statins;
one or more selected statins are applied to the cell culture;
miR-192 family member abundance in the cells is measured after the application of statins;
the first and second measurements of miR-192 family member abundance are compared; and
risk of statin-induced NOD is assessed,
wherein a decrease in miR-192 family member abundance in the cells in response to statin use is indicative of elevated risk of statin-induced NOD.

20. A kit comprising elements for the performance of the method of claim 18 or claim 19.


SEQUENCE LISTING (10 sequences)

SEQ ID NO: 1 - RNA, 21 nt, synthetic construct
  source 1..21: mol_type = other RNA
ctgacctatgaattgacagcc

SEQ ID NO: 2 - RNA, 22 nt, synthetic construct
  source 1..22: mol_type = other RNA
ctgccaattccataggtcacag

SEQ ID NO: 3 - RNA, 21 nt, synthetic construct
  source 1..21: mol_type = other RNA
tgtaacagcaactccatgtga

SEQ ID NO: 4 - RNA, 22 nt, synthetic construct
  source 1..22: mol_type = other RNA
ccagtggggctgctgttatctg

SEQ ID NO: 5 - RNA, 23 nt, synthetic construct
  source 1..23: mol_type = other RNA
tctgtcatttctttaggccaata

SEQ ID NO: 6 - DNA, 30 bp, synthetic construct
  source 1..30: mol_type = other DNA
gtgccggcttattagtgaaactggggcttg

SEQ ID NO: 7 - DNA, 32 bp, synthetic construct
  source 1..32: mol_type = other DNA
tactgagtttgagtctggggttgatttgcggc

SEQ ID NO: 8 - RNA, 110 nt, synthetic construct
  source 1..110: mol_type = other RNA
gccgagaccgagtgcacagggctctgacctatgaattgacagccagtgctctcgtctcccctctggctgccaattccataggtcacaggtatgttcgcctcaatgccagc

SEQ ID NO: 9 - RNA, 85 nt, synthetic construct
  source 1..85: mol_type = other RNA
tggttcccgccccctgtaacagcaactccatgtggaagtgcccactggttccagtggggctgctgttatctggggcgagggccag

SEQ ID NO: 10 - RNA, 110 nt, synthetic construct
  source 1..110: mol_type = other RNA
atcattcagaaatggtatacaggaaaatgacctatgaattgacagacaatatagctgagtttgtctgtcatttctttaggccaatattctgtatgactgtgctacttcaa